(19) United States
(12) Patent Application Publication
(10) Pub. No.: US 2006/0182708 A1
(43) Pub. Date: Aug. 17, 2006

(54) Prebiotically active plant extracts

(76) Inventors: Dirk Bockmuhl, Wuppertal (DE); Heide-Marie Hohne, Elsdorf (DE); Claudia Jassoy, Dusseldorf (DE); Regine Schollyssek, Mettmann (DE); Marianne Waldmann-Laue, Monheim (DE); Wolfhard Scholz, Krefeld (DE); Andrea Sattler, Dusseldorf (DE)

Correspondence Address: DANN DORFMAN HERRELL AND SKILLMAN;A PROFESSIONAL CORPORATION, 1601 MARKET STREET, SUITE 2400, PHILADELPHIA, PA, 19103-2307, US

(21) Appl. No.: 11/336,164

(22) Filed: Jan. 20, 2006

Related U.S. Application Data
(63) Continuation of application No. PCT/EP04/07708, filed on Jul. 13, 2004.

(30) Foreign Application Priority Data
Jul. 21, 2003 (DE) 103 33 245.6
Mar. 10, 2004 (DE) 10 2004 011 968.6

(51) Int. Cl. A61K 8/97; A61K 36/254; A61K 36/53; A61K 36/13; A61K 36/77

(52) U.S. Cl. 424/74; 424/728; 424/770; 424/745; 424/777

(57) ABSTRACT

The invention relates to plant extracts that have a prebiotic effect on skin, topical cosmetic and pharmaceutical compositions containing said plant extracts, and the use of said plant extracts and compositions, especially for treating the skin.

### CROSS REFERENCE TO RELATED APPLICATIONS

This application is a §365 (c) continuation application of PCT/EP2004/00708 filed 13 Jul. 2004, which in turn claims priority to DE applications 103 33 245.6 and 102 00 4011968. 6 filed 21 Jul. 2003 and 10 Mar. 2004 respectively, Each of the foregoing applications is incorporated herein by reference.

### FIELD OF THE INVENTION

The present invention relates to plant extracts that have a prebiotic effect on the skin, topical cosmetic and pharmaceutical compositions comprising said plant extracts, and methods of use thereof.

### BACKGROUND OF THE INVENTION

Dermatitis is caused by noxious bacteria, e.g., Propionibacterium acnes, which reside on the skin, but proliferate strongly under specific conditions. Uncontrolled proliferation of this particular bacteria can cause “unclean skin” or acne and under these conditions such micro organisms are classified as pathogenic. Certain fungi which reside on the skin are also considered pathogenic.

Bacterial skin flora also comprises species which do not cause harm and by virtue of their continuous growth keep the dangerous bacterial species in check thereby performing an important protective function. The coagulase-negative staphylococci S. epidermidis is representative of such a species. In general, the term “saprophyte” has been used to designate benign, desired bacterial species. However, there are also undesirable skin microorganisms that, according to the actual classification are not designated as “saprophyte” skin microorganisms, just as there are also microbes classified as “pathogenic” that are possibly desirable, so that a differentiation between “desired” and “unwanted” is not consistent with a differentiation between “saprophyte” and “pathogen”. In particular, it should also be noted, for instance, that benign skin-friendly germs can become pathogens via unchecked reproduction. The classification of the bacteria as pathogens is therefore dependent on the composition of the microbial skin flora in question.

Antibacterial agents which target skin bacteria in general, such as those used to prevent and combat acne in commercial cosmetics, not only kill unwanted skin germs but also desired skin germs thereby disturbing biological equilibrium which can give rise to a variety of undesirable consequences.

There is therefore a need for an agent that selectively encourages, on the applied skin area, the growth and/or the survival of the desired germs of the skin flora as opposed to the growth and/or the survivability of the unwanted microbes present in skin flora. Such substances are also referred to as “prebiotic”.

The prebiotic effect of substances that encourage the growth of desired intestinal bacteria are described in various publications. Ahn et al. (1990) Microbial Ecology in Health and Disease 3, 223-229, in particular also describe the use of a ginseng extract for this purpose.

To date, the only agent reported to have a prebiotic effect on skin was an oligosaccharide (promotional brochure on BioEcolia® from the Solabia Group, France), that can be selectively utilized on preferably saprophyte bacteria. It was demonstrated that the oligosaccharide described promotes the growth of Micrococcus kristinae compared with the growth of Staphylococcus aureus and of Corynebakterium xerosis.

Moreover, it was reported in EP 1050300 that a mixture of farnesol and xylitol can be used as a prebiotic substance, as this mixture acts selectively against S. aureus and therefore enables the increased reproduction of the rival S. epidermidis.

### SUMMARY OF THE INVENTION

In accordance with the present invention a variety of substances found in plant extracts that are prebiotically active on the skin or in the skin flora have been identified which can be advantageously used for treating skin. Thus, the invention encompasses compositions having beneficial effects on skin and methods of use thereof.

According to the invention, “prebiotic effect” is understood to mean that the growth and/or the survival of desired, particularly skin-friendly skin microbes or micro flora is promoted against the growth and/or the survival of the undesirable, particularly non skin-friendly skin microbes or micro flora. This can be achieved both by the active principle acting beneficially on the growth of the desired skin microbes, without directly influencing the growth of unwanted skin microbes, and also by the active principle inhibiting the growth of the unwanted skin microbes, without directly influencing the growth of the desired skin microbes. However, in a particularly preferred and particularly surprising embodiment according to the invention, the active principle acts beneficially to promote the growth of the desired skin microbes and, at the same time, inhibits the growth of the undesirable skin microbes.

According to the invention the term “skin” refers to preferably the skin itself, particularly human skin, but also the mucosa and skin adnexa, in so far that they include living cells, particularly hair follicles, hair roots, hair bulbs, the ventral epithelial layer of the nail bed (lectulus) as well as sebaceous glands and perspiratory glands.

Accordingly, the subject of the present invention is the use of prebiotically active plant extracts, principally prebiotically active on the skin, in topical cosmetic skin treatment agents to further the growth of desired skin microbes, the desired skin microbes being preferably benign and/or non-pathogenic and/or skin-friendly and/or saprophytic skin microbes and/or coagulase-negative staphylococci, particularly S. epidermidis, S. hominis, S. warneri, S. saprophyticus, S. xylosus, S. capitis or S. simulans, particularly preferably S. epidermidis or S. warneri, and/or a skin-friendly bacillus, particularly Bacillus licheniformis.

Accordingly, the invention also includes methods of using prebiotically active plant extracts, principally prebiotically active on the skin, in topical cosmetic skin treatment agents to inhibit the growth of unwanted skin microbes, the unwanted skin microbes being preferably non skin-friendly microbes and/or pathogenic microbes and/or coagulase-positive staphylococci, particularly S. aureus, or microbes selected from the group consisting of Propionibacterium acnes, Candida albicans, Malassezia furfur, Corynebacterium spp. or Peptostreptococcus spp., principally Propionibacterium acnes.

In a particularly preferred embodiment according to the invention, the prebiotically active plant extract is an extract that enhances the growth of coagulase-negative staphylococci, particularly S. epidermidis or S. warneri, and/or furthers the growth of skin-friendly bacteria, particularly Bacillus licheniformis, and at the same time inhibits the growth of Propionibacterium acnes.

Advantageously, the inventive plant extract that is prebiotically active on the skin is suitable for restoring or stabilizing the naturally occurring healthy microbial equilibrium of the skin flora.

A further object of the present invention is the use of prebiotically active plant extracts, principally prebiotically active on the skin, in topical cosmetic skin treatment agents for the treatment of bad, dry or greasy skin as well as for the treatment of fungal skin infections or dandruff, particularly the treatment of acne. The treatment can be preventative, just as for other application areas.

The invention also provides a cosmetic or pharmaceutical composition comprising a prebiotically active plant extract, principally prebiotically active on the skin, wherein the cosmetic or pharmaceutical composition is preferably a topical skin treatment agent.

The prebiotically active plant extract is preferably comprised in the composition in an amount of 0.01 to 20, particularly preferably 0.05 to 10, particularly 0.1 to 5, especially 0.5 to 2 wt. %, based on the total weight of the composition.

The inventive prebiotically active plant extract is preferably a conifer extract, particularly from the group of Pinacae, or an extract from the group of Sapindaceae, Araliaceae, Lamiaceae or Saxifragaceae or mixtures thereof.

In a preferred embodiment, the plant extract is particularly preferably an extract of Picea spp., particularly an extract of Picea excelsa (synonym Picea abies, spruce) or of Picea glauca (Norway spruce), of Pinus sp., particularly Pinus sylvestris, of Paullinia sp. (guarana), particularly Paullinia cubana, of Panax sp., particularly Panax ginseng (ginseng), of Lamium sp., particularly Lamium album (white nettle), or of Ribes sp., particularly Ribes nigrum (blackcurrant), or mixtures thereof.

The prebiotically active plant extracts can be manufactured by any method known to the expert using any plant tissue and any extracting agent. Thus, the plant extract can be extracted, for example, from the total plant, from flowers, leaves, seeds, roots and/or from the meristem of the plant.

The extracting agent used to prepare the cited plant extracts can be water, alcohols as well as their mixtures, for example. Exemplary alcohols are lower alcohols such as ethanol and isopropanol, but particularly polyhydroxy alcohols such as ethylene glycol, propylene glycol and butylene glycol, both as the sole extracting agent as well as in aqueous mixtures. Thus, plant extracts based on e.g. water/propylene glycol in proportions of 1:10 to 10:1 have proven to be particularly suitable. The extraction can be carried out by means of steam distillation, for example. Optionally, a dry extraction can also be effected.

In a preferred embodiment according to the invention, the extract of sapindaceae and particularly of guarana is a dry extract from seeds.

For the conifer extract and particularly from pinaceae, the extract according to the invention is preferably from the needles or the bark. A water/propylene glycol extract is preferred for the extract of Picea abies or Picea excelsa and a water/ethanol extract for P. glauca.

The extract from Araliaceae and particularly from ginseng is preferably a root extract.

The extract from Lamiaceae and particularly from white nettle is preferably a water/propylene glycol extract.

The extract from Saxifragaceae and particularly from blackcurrant is preferably a water/propylene glycol extract, principally from the leaves.

### DETAILED DESCRIPTION OF THE INVENTION

In accordance with the present invention, it has been discovered that certain plant extracts exert prebiotic effects on the skin, thereby enhancing the quality and appearance of skin. Thus, the invention provides such extracts and methods of use thereof, and pharmaceutical and cosmetic compositions comprising the same.

According to the invention, the plant extracts that are prebiotically active on the skin can be used in pure and also in diluted form. When they are used in diluted form, they normally comprise ca. 2-80 wt. % active substance and the solvent is the extracting agent or mixture of extracting agents used for their preparation. Depending on the choice of extracting agent, it can be preferred to stabilize the plant extract by adding a solubilizer. Suitable solubilizers are e.g. ethoxylated products of optionally hydrogenated vegetal and animal oils. Preferred solubilizers are ethoxylated mono, di and triglycerides of C8-22 fatty acids comprising 4 to 50 ethylene oxide units, e.g. ethoxylated hydrogenated castor oil, olive oil ethoxylate, almond oil ethoxylate, mink oil ethoxylate, polyoxyethylene glycol caprylic acid glycerides, polyoxyethylene glycol capric acid glycerides, polyoxyethylene glycerin mono laurate and polyoxyethylene glycol coco fatty acid glycerides.

The inventive cosmetic or pharmaceutical composition can be in any form suitable for application, e.g., a soap, a lotion, a spray, a cream, a gel, an emulsion, a cleaning fluid or cleaning milk, a deodorant, an anti-perspirant, a salve, a hair conditioner or a shampoo. The composition can also be applied in a band-aid, particularly in a gel reservoir band-aid or matrix band-aid.

The application area can be the skin of any part of the body, particularly the facial skin, the scalp, the skin on hands and feet, the skin under the arms, as well as the vaginal mucosa.

The inventive cosmetic or pharmaceutical composition can also comprise additional ingredients. In a preferred embodiment, it comprises at least one of the substances listed below. It can also comprise any desired combination of the substances listed below.

In a preferred embodiment, the inventive composition comprises at least one substance selected from vitamins, provitamins or vitamin precursors of the vitamin B group or their derivatives as well as the derivatives of 2-furanone.

The vitamin B group or the vitamin B complex include, inter alia

Vitamin B1, trivial name thiamine, chemical name 3-[(4′-amino-2′-methyl-5′-pyrimidinyl)-methyl]-5-(2-hydroxyethyl)-4-methylthiazolium chloride. Thiamine hydrochloride is preferably added in amounts of 0.05 to 1 wt. %, based on the total composition.

Vitamin B2, trivial name riboflavin, chemical name 7,8-dimethyl-10-(1-D-ribityl)-benzo[g]pteridine-2,4(3H, 10H)-dione. In its free state, riboflavin is found e.g. in whey; other riboflavin derivatives can be isolated from bacteria and yeasts. A stereoisomer of riboflavin that is also suitable according to the invention is lyoxoflavine, which can be isolated from fishmeal or liver, and has a D-arabityl group instead of D-ribityl. Riboflavin or its derivatives are preferably added in amounts of 0.05 to 1 wt. %, based on the total composition.

Vitamin B3. The compounds nicotinic acid and nicotinamide (niacinamide) are often included under this designation. According to the invention, nicotinamide is preferred and is comprised in the compositions according to the invention in amounts of 0.05 to 1 wt. % based on the total composition.

Vitamin B5 (pantothenic acid and panthenol). Preferably, panthenol is added. Useable derivatives of panthenol according to the invention are especially the esters and ethers of panthenol as well as cationic derivatized panthenols. In a further preferred embodiment of the invention, derivatives of 2-furanone with the general structural formula (I) can also be added instead of, or in addition to, pantothenic acid or panthenol.

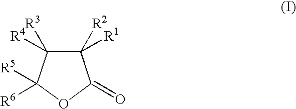

Preferred 2-furanone derivatives are those in which the substituents R1 to R6, independently of each other, represent a hydrogen atom, a hydroxy group, a methyl, methoxy, aminomethyl or hydroxymethyl group, a saturated or singly or doubly unsaturated, linear or branched C2-C4 hydrocarbon group, a saturated or singly or doubly unsaturated, linear or branched mono, di or trihydroxy C2-C4 hydrocarbon group or a saturated or singly or doubly unsaturated, linear or branched mono, di or triamino C2-C4 hydrocarbon group. Particularly preferred derivatives are also the commercially available substances dihydro-3-hydroxy-4,4-dimethyl-2(3H)-furanone with the trivial name pantolactone (Merck), 4-hydroxymethyl-γ-butyrolactone (Merck), 3,3-dimethyl-2-hydroxy-γ-butyrolactone (Aldrich) and 2,5-dihydro-5-methoxy-2-furanone (Merck), wherein all stereoisomers are expressly included. According to the invention, the greatly preferred 2-furanone derivative is pantolactone (dihydro-3-hydroxy-4,4-dimethyl-2(3H)-furanone), wherein in Formula (I) R1 stands for a hydroxy group, R2 for a hydrogen atom, R3 and R4 for a methyl group and R5 and R6 for a hydrogen atom. The stereoisomer (R)-pantolactone results from the degradation of pantothenic acid. The inventive agents preferably comprise the cited compounds of the vitamin B5 type and the 2-furanone derivatives in a total quantity of 0.05 to 10 wt. %, based on the total composition. Total quantities of 0.1 to 5 wt. % are particularly preferred.

Vitamin B6, understood not to mean a pure substance, but rather the known derivatives of 5-hydroxymethyl-2-methylpyridin-3-ol with the trivial names pyridoxine, pyridoxamine and pyridoxal. The compositions according to the invention preferably comprise Vitamin B6 in amounts of 0.0001 to 1.0 wt. %, particularly in amounts of 0.001 to 0.01 wt. %.

Vitamin B7 (biotin), also designated as Vitamin H. Biotin is (3aS,4S,6aR)-2-oxohexahydrothienol[3,4-d]-imidazol-4-valeric acid. The compositions according to the invention preferably comprise biotin in amounts of 0.0001 to 1.0 wt. %, particularly in amounts of 0.001 to 0.01 wt. %.

According to the invention, panthenol, pantolactone, nicotinamide and biotin are quite particularly preferred.

A further subject of the invention is a cosmetic or pharmaceutical skin treatment agent comprising a prebiotic plant extract and in addition, at least one further plant extract. This further plant extract can, for example, be prepared by extraction of the total plant, or also solely by extraction of flowers and/or leaves and/or seeds and/or other parts of the plant. According to the invention, preferred further plant extracts are principally extracts from the meristem i.e. from the dividable plant tissue and extracts of special plants such as green tea, hamamelis, chamomile, calendula officinalis, viola tricolor, peony, aloe vera, horse chestnut, sage, willow bark, cinnamon tree, chrysanthemum, oak bark, stinging nettle, hops, burdock root, field horsetail, hawthorn, linden flowers, almonds, sandal wood, juniper, coconut, kiwi, guavas, lime, mango, apricot, wheat, melon, orange, grapefruit, avocado, rosemary, birch, beech shoots, malva, lady's smock, common yarrow, wild thyme, thyme, lemon balm, rest-harrow, marshmallow (althaea), mallow (malva sylvestris), violets, coltsfoot, creeping cinquefoil, ginger and sweet potatoes. Algae extracts can also be advantageously added. The inventively used algae extracts originate from green algae, brown algae, red algae or blue algae (cyano bacteria). The algae used for extraction can be both of natural origin as well as from biotechnological processes and when required modified from their natural state. The modification of the organism can be genetic, by breeding or by cultivation in feedstocks enriched with selected nutrients. Preferred algae extracts originate from seaweed, blue algae, from the green algae Codium tomentosum as well as from the brown algae Fucus vesiculosus. A particularly preferred algae extract originates from blue algae of the Spirulina species that were cultivated in a magnesium-enriched medium.

Further particularly preferred plant extracts are those from spirulina, green tea, aloe vera, meristem, hamamelis, apricot, calendula officinalis, guava, sweet potatoes, lime, mango, kiwi, gherkin, malva, marshmallow and violets. The inventive agents can also comprise mixtures of several, particularly two different plant extracts as the further plant extracts.

The extracting agents used to prepare the plant extracts listed above can be those used for the preparation of the prebiotically active plant extracts, for example, water, alcohols as well as their mixtures. Exemplary preferred alcohols are lower alcohols such as ethanol and isopropanol, but particularly polyhydroxy alcohols such as ethylene glycol, propylene glycol and butylene glycol, both as the sole extracting agent as well as in aqueous mixtures. Plant extracts based on water/propylene glycol in the ratio 1:10 to 10:1 have proven particularly suitable. According to the invention, steam distillation falls among the preferred extraction processes. However, the extraction can be carried out as a dry extraction.

According to the invention, the plant extracts can be used in pure and also in diluted form. When they are used in diluted form, they normally comprise ca. 2-80 wt. % active substance and the solvent is the extracting agent or mixture of extracting agents used for their preparation. Depending on the choice of extracting agent, it can be preferred to stabilize the plant extract by adding a solubilizer. Suitable solubilizers are e.g. ethoxylated products of optionally hydrogenated vegetal and animal oils. Preferred solubilizers are ethoxylated mono, di and triglycerides of C₈₋₂₂ fatty acids comprising 4 to 50 ethylene oxide units, e.g. ethoxylated hydrogenated castor oil, olive oil ethoxylate, almond oil ethoxylate, mink oil ethoxylate, polyoxyethylene glycol caprylic acid glycerides, polyoxyethylene glycol capric acid glycerides, polyoxyethylene glycerin mono laurate and polyoxyethylene glycol coco fatty acid glycerides.

Furthermore, in addition, it can be preferred to add mixtures of several, particularly two different plant extracts to the prebiotic active plant extract.

With regard to the inventively usable plant extract, we additionally refer to extracts that are listed at the Table beginning on page 44 of the 3rd edition of the Guidelines for the Declaration of Ingredients in Cosmetics, (Leitfadens zur Inhaltsstoffdeklaration kosmetischer Mittel) published by the German Cosmetic, Toiletry, Perfumery and Detergent Association e.V. (IKW), Frankfurt.

The invention also encompasses a cosmetic or pharmaceutical skin treatment agent comprising a prebiotic plant extract and at least one MMP-1 inhibiting substance selected from photolyase and/or T4 endonuclease V, propyl gallate, precocenes, 6-hydroxy-7-methoxy-2,2-dimethyl-(2H)-1-benzopyran, 3,4-dihydro-6-hydroxy-7-methoxy-2,2-dimethyl-(2H)-1-benzopyran (available as the commercial product Lipochroman 6® from Lipotec SA) and their mixtures. Precocenes are chromene derivatives that occur naturally in plants and are known hormones (The Merck Index, 12th edition, Merck & Co. 1996). The MMP-1 inhibiting action of these substances is described in the German disclosure DE 100116016 A1. They are preferably added in amounts of 0.1 to 5 wt. %, preferably 0.5 to 2 wt. %, each based on the total composition.

In a particularly preferred embodiment, the inventive skin treatment agents additionally comprise at least one retinol (vitamin A1) ester of a C₂₋₁₈ carboxylic acid. Preferred retinol esters are retinyl acetate and retinyl palmitate, retinyl palmitate being particularly preferred. The retinol esters are added in amounts of 0.1 to 5 wt. %, preferably 0.5 to 2 wt. %, each based on the total composition.

In a further preferred embodiment, the inventive skin treatment agents, particularly when used as an emulsion or surfactant-containing solution, primarily as detergents, comprise at least one surface-active substance as the emulsifier or dispersion agent. Emulsifiers act at the interphase to produce water or oil-stable adsorption layers that protect the dispersed droplets against coalescence and thereby stabilize the emulsion. Thus, emulsifiers, like surfactants are composed of hydrophobic and hydrophilic molecular moieties. Hydrophilic emulsifiers preferably form O/W emulsions and hydrophobic emulsifiers preferably form W/O emulsions. W/O emulsions that are stabilized in the absence of hydrophilic emulsifiers are disclosed in DE 19816665 A1 and DE 19801593 A1. An emulsion is understood to mean a dispersion of a liquid in the form of droplets in another liquid using an energy input to afford interphases stabilized with surfactants. The choice of this emulsifying surfactant or emulsifier depends on the materials being dispersed and the respective external phase as well as the fineness of the emulsion.

Exemplary emulsifiers usable according to the invention are

Addition products of 4 to 30 moles ethylene oxide and/or 0 to 5 moles propylene oxide on linear C₈-C₂₂-fatty alcohols, on C₁₂-C₂₂-fatty acids and on C₈-C₁₅-alkylphenols,

C₁₂-C₂₂ fatty acid mono and diesters of addition products of 1 to 30 moles ethylene oxide on C₃-C₆ polyols, particularly on glycerin,

ethylene oxide and polyglycerin addition products on methylglucoside fatty acid esters, fatty acid alkanolamides and fatty acid glucamides,

C₈-C₂₂ alkyl mono and oligoglycosides and their ethoxylated analogs, wherein the degrees of oligomerization are 1.1 to 5, particularly 1.2 to 2.0, and glucose as the sugar component, is preferred,

mixtures of alkyl(oligo) glucosides and fatty alcohols, for example the commercial product Montanov®68,

Addition products of 5 to 60 moles ethylene oxide on castor oil and hydrogenated castor oil,

partial esters of polyols containing 3-6 carbon atoms with C₈-C₂₂ fatty acids,

sterols (sterine). Sterols are understood to mean a group of steroids, which carry a hydroxyl group on carbon atom 3 of the steroid skeleton and are isolated from both animal tissue (zoosterols) and vegetal fats (phytosterols). Examples of zoosterols are cholesterol and lanosterol. Examples of suitable phytosterols are beta-sitosterol, stigmasterol, campesterol and ergosterol. Sterols, the so-called mycosterols, are also isolated from fungi and yeasts.

phospholipids, principally the glucose-phospholipids, which are obtained e.g. as lecithins or phosphatidyl cholines from e.g. egg yolk or plant seeds (e.g. soya beans),

fatty acid esters of sugars and sugar alcohols such as sorbitol,

polyglycerin and polyglycerin derivatives, preferably polyglyceryl-2-dipolyhydroxy stearate (commercial product Dehymuls® PGPH) and polyglyceryl-3-diisostearate (commercial product Lameform® TGI),

linear and branched C₈-C₃₀ fatty acids and their Na, K, ammonium, Ca, Mg and Zn salts.

The inventive compositions preferably comprise the emulsifiers in quantities of 0.1 to 25 wt. %, particularly 0.5-15 wt. %, based on the total composition.

In a particularly preferred embodiment, at least one non-ionic emulsifier with a HLB value of 8 and below is comprised (according to the definition of HLB value shown in the Römpp-Lexikon Chemie (Eds.: J. Falbe, M. Regitz), 10th edition, Georg Thieme Verlag Stuttgart, New York, (1997), page 1764). Exemplary suitable emulsifiers of this type are compounds of the general formula R¹—O—R², in which R¹ is a primary linear alkyl, alkenyl or acyl group having 20 to 30 carbon atoms and R² is hydrogen, a group of formula —(CₙH₂ₙO)ₓ—H with x=1 or 2 and n=2 to 4 or a polyhydroxyalkyl group with 4 to 6 carbon atoms and 2 to 5 hydroxy groups. A particularly preferred emulsifier of formula R¹—O—R² is a behenyl or erucyl derivative, in which R¹ represents a primary linear alkyl, alkenyl or acyl group having 22 carbon atoms.

Further preferred suitable emulsifiers with a HLB value of 8 and below are the addition products of 1 or 2 moles ethylene oxide or propylene oxide on behenyl alcohol, erucyl alcohol, arachidyl alcohol or also on behenic acid or erucic acid. Monoesters of C₁₆-C₃₀ fatty acids with polyols such as e.g. pentaerythreitol, trimethylol propane, diglycerin, sorbitol, glucose or methylglucose are also suitable and preferred. Examples of such products are e.g. sorbitol monobehenate or pentaerythreitol monoerucate.

In another similarly particularly preferred embodiment, at least one ionic emulsifier, selected from anionic, zwitterionic, ampholytic and cationic emulsifiers, is comprised Preferred anionic surfactants are alkyl sulfates, alkyl polglycol ether sulfates and ether carboxylic acids with 10 to 18 C atoms in the alkyl group and up to 12 glycol ether groups in the molecule sulfosuccinic acid mono and dialkyl esters with 8 to 18 C atoms in the alkyl group and sulfosuccinic acid mono-alkylpolyoxyethyl esters with 8 to 18 C atoms in the alkyl group and 1 to 6 oxyethylene groups, monoglyceride sulfates, alkyl and alkenyl ether phosphates as well as condensates of protein and fatty acids. Zwitterionic emulsifiers carry at least a quaternary ammonium group and at least one —COO⁻— or SO₃⁻ group in the molecule. Particularly suitable zwitterionic emulsifiers are the so-called betaines such as N-alkyl-N,N-dimethylammonium glycinates, N-acyl-aminopropyl-N,N-dimethylammonium glycinates and 2-alkyl-3-carboxymethyl-3-hydroxyethyl-imidazolines, each having 8 to 18 carbon atoms in the alkyl or acyl group, as well as cocoacylaminoethylhydroxyethylcarboxymethyl glycinate.

Ampholytic emulsifiers comprise, apart from a C₈-C₂₄ alkyl or acyl group, at least one free amino group and at least one COOH or SO₃H group in the molecule, and are able to form internal salts. Examples of suitable ampholytic emulsifiers are N-alkylglycines, N-alkylaminopropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids each with about 8 to 24 carbon atoms in the alkyl group.

The ionic emulsifiers are comprised in quantities of 0.01 to 5 wt. %, preferably 0.05 to 3 wt. % and particularly preferably from 0.1 to 1 wt. %, based on the total composition.

In addition, the inventive compositions can comprise foaming, non-ionic, zwitterionic, anionic and cationic surfactants.

Examples of non-ionic surfactants are

alkoxylated fatty acid alkyl esters of the formula R¹CO—(OCH₂CHR²)ₓOR³ in which R¹CO stands for a linear or branched, saturated and/or unsaturated acyl group with 6 to 22 carbon atoms, R² for hydrogen or methyl, R³ for linear or branched alkyl groups with 1 to 4 carbon atoms and x for numbers from 1 to 20,

addition products of ethylene oxide to fatty acid alkanolamides and fatty amines,

fatty acid N-alkylglucamides,

C₈-C₂₂ alkylamine-N-oxides,

alkyl polyglycosides corresponding to the general formula RO—(Z)ₓ wherein R stands for a C₈-C₁₆ alkyl, Z for sugar and x for the number of sugar units. The alkyl polyglycosides used according to the invention may simply comprise a defined alkyl group R. However normally, these compounds are manufactured from natural fats and oils or mineral oils. In which case, the alkyl groups Rare present as mixtures corresponding to the starting compounds or to each of the compounds worked up. Alkyl polyglycosides are particularly preferred, in which R is essentially from C₈- and C₁₀-alkyl groups, is essentially from C₁₂- and C₁₄-alkyl groups, is essentially from C₈-bis C₁₆-alkyl groups or is essentially from C₁₂-bis C₁₆-alkyl groups.

Any mono or oligosaccharide can be added as the sugar building block Z. Normally, sugars having 5 or 6 carbon atoms as well as the corresponding oligosaccharides are added, for example, glucose, fructose, galactose, arabinose, ribose, xylose, lyxose, allose, altrose, mannose, gulose, idose, talose and sucrose. Preferred sugar building blocks are glucose, fructose, galactose, arabinose and sucrose; glucose is particularly preferred. The inventively usable alkyl polyglycosides comprise an average of 1.1 to 5, preferably 1.1 to 2.0, particularly preferably 1.1 to 1.8 sugar units. The alkoxylated homologs of the cited alkyl polyglycosides can also be used according to the invention. These homologs can comprise on average up to 10 ethylene oxide and/or propylene oxide units per alkyl glycoside unit.

Zwitterionic surfactants are designated as those surface-active compounds that carry at least one quaternary ammonium group and at least one —COO⁽⁻⁾ or SO₃⁽⁻⁾ (group in the molecule. Particularly suitable zwitterionic surfactants are the so-called betaines such as the N-alkyl-N,N-dimethylammonium glycinates, for example the cocoalkyldimethylammonium glycinate, N-acylaminopropyl-N,N-dimethylammonium glycinates, for example the cocoacylaminopropyldimethylammonium glycinate, and 2-alkyl-3-carboxymethyl-3-hydroxyethyl imidazolines with 8 to 18 carbon atoms in each of the alkyl or acyl groups, as well as cocoacylaminoethylhydroxyethylcarboxymethyl glycinate. A preferred zwitterionic surfactant is the fatty acid amide derivative, known under the INCI name cocoamidopropyl betaine.

Suitable anionic surfactants for the inventive preparations are all anionic surface-active materials that are suitable for use on the human body. They are characterized by a water solubilizing anionic group, such as e.g. a carboxylate, sulfate, sulfonate or phosphate group and a lipophilic alkyl group containing about 8 to 30 C atoms. In addition, the molecule may contain glycol or polyglycol ether groups, ester, ether and amide groups as well as hydroxyl groups. Exemplary suitable foaming anionic surfactants are, each in the form of the sodium, potassium and ammonium as well as the mono, di and trialkanolammonium salts with 2 to 4 carbon atoms in the alkanol group,

acylglutamates of Formula (II), 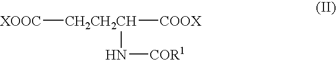
in which R¹CO stands for a linear or branched acyl group with 6 to 22 carbon atoms and 0, 1, 2 or 3 double bonds and X for hydrogen, an alkali and/or alkaline earth metal, ammonium, alkylammonium, alkanolammonium or glucammonium, for example acylglutamates that derive from fatty acids having 6 to 22, preferably 12 to 18 carbon atoms, such as, for example C_{12/14}- or C_{12/18}-coco fatty acid, lauric acid, myristic acid, palmitic acid and/or stearic acid, particularly sodium N-cocoyl and sodium N-stearoyl-L-glutamate,

esters of a hydroxy-substituted di or tricarboxylic acid of the general formula (III), 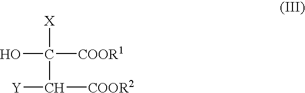
in which X═H or is a —CH₂COOR group, Y═H or —OH, with the proviso that Y═H if X═—CH₂COOR, R, R¹ and R², independently of each other signify a hydrogen atom, an alkali or alkaline earth metal cation, an ammonium group, the cation of an ammonium organic base or a group Z which derives from a polyhydroxylated organic compound selected from the group of etherified (C₆-C₁₈)-alkylpolysaccharides having 1 to 6 monomeric saccharide units and/or the etherified aliphatic (C₆-C₁₆)-hydroxyalkyl polyols having 2 to 16 hydroxyl groups, with the proviso that at least one of the groups R, R¹ and R² is a group Z,

esters of the sulfosuccinic acid of the general formula (IV), 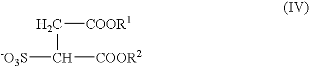
in which R¹ and R², independently of one another signify a hydrogen atom, an alkali or alkaline earth metal cation, an ammonium group, the cation of an ammonium organic base or a group Z which derives from a polyhydroxylated organic compound selected from the group of etherified (C₆-C₁₈)-alkylpolysaccharides having 1 to 6 monomeric saccharide units and/or the etherified aliphatic (C₆-C₁₆)-hydroxyalkyl polyols having 2 to 16 hydroxyl groups, with the proviso that at least one of the groups R1 or R2 is a group Z,
sulfosuccinic acid mono and dialkyl esters with 8 to 24 carbon atoms in the alkyl group and sulfosuccinic acid mono-alkylpolyoxyethyl esters with 8 to 24 C atoms in the alkyl group and 1 to 6 ethoxy groups,
esters of tartaric acid and citric acid with alcohols, which represent the addition products of about 2-15 molecules of ethylene oxide and/or propylene oxide on fatty alcohols with 8 to 22 C atoms,
linear and branched fatty acids with 8 to 30 C atoms (soaps),
ether carboxylic acids of the formula R—O—(CH₂—CH₂O)ₓ—CH₂—COOH, in which R is a linear alkyl group with 8 to 30 C atoms and x=0 or 1 to 16,
acyl sarcosinates with a linear or branched acyl group having 6 to 22 carbon atoms and 0, 1, 2 or 3 double bonds,

acyl taurates with a linear or branched acyl group having 6 to 22 carbon atoms and 0, 1, 2 or 3 double bonds,
acyl isethionates with a linear or branched acyl group having 6 to 22 carbon atoms and 0, 1, 2 or 3 double bonds,
linear alkane sulfonates with 8 to 24 C atoms,
linear alpha-olefin sulfonates with 8 to 24 C atoms
alpha-sulfo fatty acid methyl esters of fatty acids with 8 to 30 C atoms,
alkyl sulfates and alkyl polyglycol ether sulfates of the formula R—O(CH₂—CH₂O)₂—SO₃X, in which R is a preferably linear alkyl group having 8 to 30 carbon atoms, particularly preferably 8 to 18 carbon atoms, z=0 or 1 to 12, particularly preferably 3, and X is a sodium, potassium, magnesium, zinc, ammonium ion or a monoalkanol-, dialkanol- or trialkanolammonium ion having 2 to 4 carbon atoms in the alkanol groups, wherein a particularly preferred example is zinc cocoyl ether sulfate having an ethoxylation degree of z=3,
mixtures of surface-active hydroxy sulfonates according to DE-A-37 25 030,
sulfated hydroxyalkyl polyethylenes and/or hydroxyalkylene propylene glycol ethers according to DE-A-37 23 354,
sulfonated unsaturated fatty acids with 8 to 24 C atoms and 1 to 6 double bonds according to DE-A-39 26 344,
alkyl- and/or alkenyl ether phosphates of Formula (V), 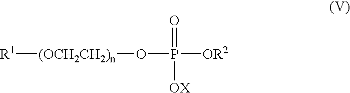
   in which R¹ preferably stands for an aliphatic hydrocarbon radical with 8 to 30 carbon atoms, R² stands for hydrogen, a (CH₂CH₂O)ₙR¹ group or X, n for numbers between 1 and 10 and X for hydrogen, an alkali or alkaline earth metal or NR³R⁴R⁵R⁶, with R³ to R⁶, independently of each other standing for a C₁, to C₄ hydrocarbon group,

sulfated fatty acid alkylene glycol esters of Formula R⁷CO(AlkO)ₙSO₃M in which R⁷CO stands for a linear or branched, aliphatic, saturated and/or unsaturated acyl radical with 6 to 22 carbon atoms, Alk for CH₂CH₂, CHCH₃CH₂ and/or CH₂CHCH₃, n for numbers from 0.5 to 5 and M for a cation, like those described in DE-OS 197 36 906.5

monoglyceride sulfates and monoglyceride ether sulfates of Formula (VI),

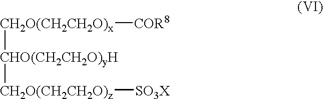

in which R⁸CO stands for a linear or branched acyl group with 6 to 22 carbon atoms, the sum of x, y and z is 0 or stands for numbers between 1 and 30, preferably 2 to 10, and X stands for an alkali or alkaline earth metal. In the context of the invention, typical examples of suitable monoglyceride (ether) sulfates are the reaction products of lauric acid monoglyceride, cocoa fatty acid monoglyceride, palmitic acid monoglyceride, stearic acid monoglyceride, oleic acid monoglyceride and tallow fatty acid monoglyceride as well as their ethylene oxide adducts with sulfur trioxide or chlorosulfonic acid in the form of their sodium salts. Preferably, monoglyceride sulfates of Formula (VI) are added, in which R⁸CO stands for a linear acyl group with 8 to 18 carbon atoms.

In a further preferred embodiment, the inventive cosmetic compositions comprise at least one organic or mineral or modified mineral light stabilizer. The light stabilizers are liquid or crystalline substances at room temperature which are able to absorb UV radiation and emit the resulting energy in the form of longer wavelength radiation, for example as heat. One differentiates between UVA-filters and UVB-filters. Naturally, the UV-A and UV-B filters can also be added as mixtures. According to the invention, it is preferred to add mixtures of filters.

The organic UV-filters used according to the invention are selected from derivatives of dibenzoyl methane, cinnamic acid esters, diphenylacrylic acid esters, benzophenone, camphor, p-aminobenzoic acid esters, o-aminobenzoic acid esters, salicylic acid esters, benzimidazoles, symmetrical or unsymmetrical substituted 1,3,5-triazines, monomeric and oligomeric 4,4-diarylbutadienecarboxylic acid esters and -carboxylic acid amides, ketotricyclo(5.2.1.0)decane, benzalmalonic acid esters as well as any mixtures of the cited components. The organic UV-filters can be oil-soluble or water-soluble. According to the invention, particularly preferred oil-soluble UV-filters are 1-(4-tert.-butylphenyl)-3-(4′-methoxyphenyl)propane-1,3-dione (Parsol® 1789), 1-phenyl-3-(4′-isopropylphenyl)-propane-1,3-dione, 3-(4′-methylbenzylidene)-D,L-camphor, 4-(dimethylamino)-benzoic acid 2-ethylhexyl ester, 4-(dimethylamino)benzoic acid 2-octyl ester, 4-(dimethylamino)-benzoic acid amyl ester, 4-methoxycinnamic acid 2-ethylhexyl ester, 4-methoxycinnamic acid propyl ester, 4-methoxycinnamic acid isopentyl ester, 2-cyano-3,3-phenylcinnamic acid 2-ethylhexyl ester (octocrylene), salicylic acid 2-ethylhexyl ester, salicylic acid 4-isopropylbenzyl ester, salicylic acid homomenthyl ester (3,3,5-trimethyl-cyclohexyl salicylate), 2-hydroxy-4-methoxybenzophenone, 2-hydroxy-4-methoxy-4′-methylbenzophenone, 2,2′-dihydroxy-4-methoxybenzophenone, 4-methoxybenzmalonic acid di-2-ethylhexyl ester, 2,4,6-trianilino-(p-carbo-2′-ethyl-1′-hexyloxy)-1,3,5-triazine (octyl triazone) and dioctyl butamido triazone (Uvasorb® HEB) as well as any mixtures of the cited components.

Preferred water-soluble UV-filters are 2-phenylbenzimidazole-5-sulfonic acid and its alkali-, earth alkali-, ammonium-, alkylammonium-, alkanolammonium- and glucammonium salts; sulfonic acid derivatives of benzophenones, preferably 2-hydroxy-4-methoxybenzophenone-5-sulfonic acid and its salts; sulfonic acid derivatives of 3-benzylidenecamphor, as for example 4-(2-oxo-3-bornylidenemethyl)benzene sulfonic acid and 2-methyl-5-(2-oxo-3-bornylidene) sulfonic acid and their salts.

Some of the oil-soluble UV-filters can serve as solvents or solubilizers for other UV-filters. Thus, for example, solutions of the UV-filter 1-(4-tert.-butylphenyl)-3-(4′methoxyphenyl)propane-1,3-dione (e.g. Parsol® 1789) can be prepared in various UV-B filters. In a further preferred embodiment, the inventive compositions therefore comprise 1-(4-tert.-butylphenyl)-3-(4′-methoxyphenyl)propane-1,3-dione in combination with at least one UV-B filter, selected from 4-methoxycinnamic acid 2-ethylhexyl ester, 2-cyano-3,3-phenylcinnamic acid 2-ethylhexyl ester, salicylic acid 2-ethylhexyl ester and 3,3,5-trimethylcyclohexyl salicylate. In these combinations the ratio by weight of the UV-B filter to the 1-(4-tert.-butylphenyl)-3-(4′-methoxyphenyl)propane-1,3-dione is between 1:1 and 10:1, preferably between 2:1 and 8:1, the molar ratio lying correspondingly between 0.3 and 3.8, preferably between 0.7 and 3.0.

The inventively preferred inorganic light stabilizer pigments are finely divided or colloidally dispersed metal oxides and metal salts, e.g. titanium dioxide, zinc oxide, iron oxide, aluminum oxide, cerium oxide, zirconium oxide, silicates (talc) and barium sulfate. Here, the particles should have a mean diameter of less than 100 nm, preferably between 5 and 50 nm and especially between 15 and 30 nm, so-called nanopigments. They can be spherical, however elliptical or other shaped particles can also be used. The pigments can also be surface treated, i.e. hydrophilized or hydrophobized. Typical examples are coated titanium dioxides, such as, for example Titandioxid T 805 (Degussa) or Eusolex® T2000 (Merck). Hydrophobic coating agents preferably include trialkoxy octylsilanes or Simethicones. Titanium dioxide and zinc oxide are particularly preferred.

In addition, it has proven particularly advantageous that the inventive skin treatment agents comprise at least one protein hydrolyzate or the derivative thereof. According to the invention, the added protein hydrolyzates can be of either vegetal or animal origin. Animal protein hydrolyzates are, for example, elastin, collagen, keratin, silk and milk protein hydrolyzates, which can also be present in the form of their salts. According to the invention, protein hydrolyzates of vegetal origin, e.g. soya, wheat, almond, pea, potatoes and rice protein hydrolyzates, are preferred. Corresponding commercial products include for example, DiaMin (E (Diamalt), Gluadin® (Cognis), Lexein® (Inolex) and Crotein® (Croda).

The protein hydrolyzate described above can be substituted with amino acid mixtures, or individual amino acids as well as their physiologically compatible salts. Preferred amino acids for this purpose include for example, glycine, serine, threonine, cysteine, asparagine, glutamine, pyroglutamic acid, alanine, valine, leucine, isoleucine, proline, tryptophan, phenylalanine, methionine, tyrosine, asparaginic acid, glutamic acid, lysine, arginine and histidine as well as the zinc salts and the acid addition salts of these amino acids.

Likewise, it is possible to add derivatives of protein hydrolyzates, e.g. in the form of their fatty acid condensation products, e e.g. Lamepon® (Cognis), Gluadin® (Cognis), Lexein® (Inolex), Crolastin® or Crotein® (Croda).

According to the invention, cationic protein hydrolyzates may also be employed which are obtainable from a variety of biological sources, including without limitation, animals, plants, marine life forms or from protein hydrolyzates of biotechnological origin. Preferred cationic protein hydrolyzates possess a base protein content of molecular weight of 100 to 25000 daltons, preferably 250 to 5000 daltons. In this context, cationic protein hydrolyzates are understood to include quaternized amino acids and their mixtures. Moreover, the cationic protein hydrolyzates can also be further derivatized. Typical examples of inventive cationic protein hydrolyzates and derivatives thereof are available commercially and include those cited in the “International Cosmetic Ingredient Dictionary and Handbook”, (seventh edition 1997, The Cosmetic, Toiletry, and Fragrance Association 1101 17th Street, N. W., Suite 300, Washington, D.C. 20036-4702). See for example, cocodimonium hydroxypropyl hydrolyzed collagen, cocodimonium hydroxypropyl hydrolyzed casein, steardimonium hydroxypropyl hydrolyzed collagen, steardimonium hydroxypropyl hydrolyzed hair keratin, lauryldimonium hydroxypropyl hydrolyzed keratin, cocodimonium hydroxypropyl hydrolyzed rice protein, cocodimonium hydroxypropyl hydrolyzed silk, cocodimonium hydroxypropyl hydrolyzed soy protein, cocodimonium hydroxypropyl hydrolyzed wheat protein, cocodimonium hydroxypropyl silk amino acids, hydroxypropyl arginine lauryl/myristyl ether HCl, hydroxypropyltrimonium gelatin. Cationic protein hydrolyzates and derivatives obtained from plants are quite particularly preferred.

The compositions according to the invention comprise the protein hydrolyzates and their derivatives or the amino acids and their derivatives in quantities of 0.01 to 10 wt. %, based on the total composition. Quantities of 0.1 to 5 wt. %, particularly 0.1 to 3 wt. %, are quite particularly preferred.

In addition, it has proven particularly advantageous to include at least one mono, oligo or polysaccharide or the derivatives thereof in the inventive skin treatment agents described herein.

According to the invention, suitable monosaccharides include, without limitation, glucose, fructose, galactose, arabinose, ribose, xylose, lyxose, allose, altrose, mannose, gulose, idose and talose, the desoxysugar fucose and rhamnose as well as amino sugars such as e.g. glucosamine or galactosamine. Glucose, fructose, galactose, arabinose and fucose are preferred; glucose is particularly preferred.

According to the invention, suitable oligosaccharides are composed of two to ten monosaccharide units, e.g. saccharose, lactose or trehalose. Saccharose is a particularly preferred oligosaccharide. The use of honey, which comprises mainly glucose and saccharose, is also particularly preferred.

According to the invention, suitable polysaccharides are composed of more than ten monosaccharide units. Preferred polysaccharides are the starches based on α-D-glucose units as well as starch degradation products e.g., amylose, amylopectin and dextrin. According to the invention, chemically and/or thermally modified starches, e.g. hydroxypropyl starch phosphate, dihydroxypropyl distarch phosphate or the commercial product Dry Flo® are particularly advantageous. Dextrans as well as their derivatives, e.g. dextran sulfate, are also preferred. Non-ionic cellulose derivatives, such as methyl cellulose, hydroxypropyl cellulose or hydroxyethyl cellulose, as well as cationic cellulose derivatives, e.g. the commercial products Celquat and Polymer JR®, and preferably Celquat® H 100, Celquat® L 200 and Polymer JR® 400 (Polyquaternium-10) as well as Polyquaternium-24, are similarly preferred. Further preferred examples are polysaccharides from fucose units, e.g. the commercial product Fucogel®. Polysaccharides based on amino sugar units, particularly chitins and their deacetylated derivatives, the chitosans, and mucopolysaccharides are particularly preferred. The mucopolysaccharides, preferred according to the invention, include hyaluronic acid and its derivatives, e.g. sodium hyaluronate or dimethylsilanol hyaluronate, as well as chondroitin and its derivatives, e.g. chondroitin sulfate.

In a particularly advantageous embodiment, the inventive skin treatment agents comprise at least one film forming, emulsion stabilizing, thickening or adhesive polymer, selected from naturally occurring and synthetic polymers which can be cationic, anionic, amphoterically charged or non-ionic.

Cationic, anionic as well as non-ionic polymers are preferred according to the invention.

Preferred cationic polymers include polysiloxanes having quaternary groups, e.g. the commercial products Q2-7224 (Dow Corning), Dow Corning® 929 Emulsion (with amodimethicone), SM-2059 (General Electric), SLM-55067 (Wacker) as well as Abil®-Quat 3270 and 3272 (Th. Goldschmidt).

Preferred anionic polymers, which can support the action of the inventively used active principle, comprise carboxylate- and/or sulfonate groups and for example acrylic acid, methacrylic acid, crotonic acid, maleic anhydride and 2-acrylamido-2-methylpropane sulfonic acid as monomers. Here, the acidic groups may be fully or partially present as sodium, potassium, ammonium, mono or triethanolammonium salts. Preferred monomers are 2-acrylamido-2-methylpropane sulfonic acid and acrylic acid. Quite particularly preferred anionic polymers comprise 2-acrylamido-2-methylpropane sulfonic acid as the sole monomer or comonomer, wherein the sulfonic acid group can be totally or partially present as the salt. In this embodiment, it is preferred to use copolymers of at least one anionic monomer and at least one non-ionic monomer. Regarding the anionic monomers, reference is made to the abovementioned substances. Preferred non-ionic monomers are acrylamide, methacrylamide, acrylic acid esters, methacrylic acid esters, vinyl pyrrolidone, vinyl ethers and vinyl esters. Preferred anionic copolymers are acrylic acid-acrylamide copolymers and particularly polyacrylamide copolymers with monomers that contain sulfonic acid groups. A particularly preferred anionic copolymer consists of 70 to 55 mole % acrylamide and 30 to 45 mole % 2-acrylamido-2-methylpropane sulfonic acid, wherein the sulfonic acid groups may be fully or partially present as the sodium, potassium, ammonium, mono or triethanolammonium salt. This copolymer can also be crosslinked, wherein the preferred crosslinking agents include polyolefinic unsaturated compounds such as tetraallyloxyethane, allyl sucrose, allyl pentaerythritol and methylene bisacrylamide. Such a polymer is comprised in the commercial product Sepigel® 305 from the SEPPIC company. In the context of the inventive teaching, the use of this compound has proved to be particularly advantageous. The sodium acryloyl dimethyl taurate copolymers, commercialized as a compound with isohexadecane and polysorbate 80, under the trade name Simulgel®600, have also proved to be particularly effective according to the invention.

Further particularly preferred anionic homo and copolymers are uncrosslinked and crosslinked polyacrylic acids. Here the preferred crosslinking agents can be allyl ethers of pentaerythritol, of sucrose and of propylene. The commercial products Carbopol® are examples of such compounds A particularly preferred anionic copolymer comprises the monomers 80-98% of an unsaturated, optionally substituted C₃₋₆ carboxylic acid or its anhydride as well as 2-20% of an optionally substituted acrylic acid ester of saturated C₁₀₋₃₀ carboxylic acids, wherein the copolymer can be crosslinked with the abovementioned crosslinking agents. The corresponding commercial products are Pemulen® and the Carbopol® types 954, 980, 1342 and ETD 2020 (B.F. Goodrich).

Suitable non-ionic polymers include polyvinyl alcohols that can be partially saponified, e.g. the commercial products Mowiol® as well as vinyl pyrrolidone/vinyl ester copolymers and polyvinyl pyrrolidones that are marketed e.g. under the trade name Luviskol® (BASF).

In a further preferred embodiment of the invention, the action of the inventive agent can be further optimized by means of fats. Examples of suitable fats are:
vegetal oils, such as sunflower oil, olive oil, soya oil, rapeseed oil, almond oil, jojoba oil, orange oil, wheat germ oil, peach stone oil and the liquid parts of coconut oil.
liquid paraffin oils, isoparaffin oils and synthetic hydrocarbons, e.g. 1,3-di-(2-ethylhexyl)-cyclohexane (Cetiol® S) or polydecene,
di-n-alkyl ethers having a total of 12 to 36, particularly 12 to 24 carbon atoms, e.g. di-n-octyl ether (Cetiol® OE), n-hexyl n-octyl ether and n-octyl n-decyl ether.
fatty acids, particularly linear and/or branched, saturated and/or unsaturated C₈₋₃₀-fatty acids. C₁₀₋₂₂-fatty acids are preferred. Examples are the iso stearic acids and iso palmitic acids such as the fatty acids marketed under the trade name Edenor®. Further typical examples of such fatty acids are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linolic acid, linolenic acid, elaeostearic acid, arachidonoic acid, gadoleic acid, behenic acid and erucic acid as well as their technical mixtures. Usually, the fatty acid fractions obtainable from coconut oil and palm oil are particularly preferred; the addition of stearic acid is especially preferred.
fatty alcohols, particularly saturated, mono or polyunsaturated, branched or linear fatty alcohols with 6 to 30, preferably 10 to 22 and quite particularly preferably 12 to 22 carbon atoms. In the scope of the invention, decanol, octanol, octenol, dodecenol, decenol, octadienol, dodecadienol, decadienol, oleyl alcohol, erucic alcohol, ricinolyl alcohol, stearyl alcohol, isostearyl alcohol, cetyl alcohol, lauryl alcohol, myristyl alcohol, arachidyl alcohol, capryl alcohol, caprin alcohol, linoleyl alcohol, linolenyl alcohol and behenyl alcohol, as well as the Guerbet alcohols, e.g. 2-ethylhexanol are suitable examples, this listing being intended as exemplary and not limiting.
Ester oils, i.e. esters of C₆₋₃₀— fatty acids with C₂₋₃₀-fatty alcohols. Monoesters of fatty acids with alcohols having 2 to 24 carbon atoms are preferred. The abovementioned substances can be used as the alcohol and acid components of the ester oils. According to the invention, isopropyl myristate, isononanoic acid C₁₆₋₁₈-alkyl ester, 2-ethylhexyl palmitate, stearic acid 2-ethylhexyl ester, cetyl oleate, glycerin tricaprylate, coco fatty alcohol caprinate/-caprylate, n-butyl stearate, oleyl erucate, isopropyl palmitate, oleyl oleate, lauric acid hexyl ester, di-n-butyl adipate, myristyl myristate, cetearyl isononanoate and oleic acid decyl ester are particularly preferred.
hydroxycarboxylic acid alkyl esters, wherein fully esterified glycolic acid, lactic acid, malic acid, tartaric acid or citric acid are preferred, but also esters of β-hydroxypropionic acid, tartronic acid, D-gluconic acid, saccharic acid, mucic acid or glucoronic acid are suitable, and particularly preferably the esters of C₁₂-C₁₅ fatty alcohols, e.g. the commercial products Cosmacol® of Enichem, Augusta Industriale,
Dicarboxylic acid esters such as di-n-butyl adipate, di-(2-ethylhexyl) adipate, di-(2-ethylhexyl) succinate und di-isotridecyl acetate as well as diol esters such as ethylene glycol dioleate, ethylene glycol di-isotridecanoate, propylene glycol di(2-ethylhexanoate), propylene glycol di-isostearate, propylene glycol di-pelargonate, butanediol di-isostearate, neopentyl glycol dicaprylate,
symmetrical, unsymmetrical or cyclic esters of carbon dioxide with fatty alcohols, e.g. glycerin carbonate or dicaprylyl carbonate (Cetiol® CC),
mono, di and trifatty acid esters of saturated and/or unsaturated linear and/or branched fatty acids with glycerin, e.g. Monomuls® 90-018, Monomuls® 90-L12 or Cutina® MD,
Waxes, particularly insect waxes such as beeswax and bumblebee wax, plant waxes such as candelilla wax and carnauba wax, fruit waxes, ozokerite, microwax, ceresin, paraffin, triglycerides of saturated and optionally hydroxylated C₁₆₋₃₀ fatty acids, such as e.g. hydrogenated triglyceride fats (hydrogenated palm oil, hydrogenated coconut oil, hydrated castor oil), glyceryl tribehenate or glyceryl tri(12-hydroxystearate), synthetic total esters of fatty acids and glycols (e.g. Syncrowachs®) or polyols with 2 to 6 carbon atoms, esters of optionally hydroxylated C₂₋₄ carboxylic acids with lanolin alcohols and C₁₂₋₁₈ fatty alcohols, cholesterol esters or lanosterol esters of C₁₀₋₃₀ fatty acids, ethoxylated C₁₂₋₂₀ fatty acid glycol esters, fatty acid monoalkanolamides with a C₁₂₋₂₂ acyl group and a C₂₋₄ alkanol group, synthetic fatty acid-fatty alcohol esters, e.g. stearyl stearate or cetyl palmitate as well as ester waxes from fatty acids of natural origin and synthetic C₂₀₋₄₀ fatty alcohols (INCI designation C20-40 Alkyl Stearate),
silicone compounds selected from decamethylcyclopentasiloxane, dodecamethylcyclohexasiloxane and silicone polymers, which can be optionally crosslinked, e.g. polydialkylsiloxanes, Polyalkylarylsiloxanes, ethoxylated polydialkylsiloxanes, preferably the substances with the INCI designation Dimethicone Copolyol, as well as polydialkylsiloxane that comprise amine and/or hydroxy groups.

The fat content is 0.1-50 wt. %, preferably 0.1-20 wt. % and particularly preferably 0.1-15 wt. %, in each case based on the total weight of the composition.

In a further preferred embodiment of the invention, the skin treatment agent comprises at least one α-hydroxycarboxylic acid or α-ketocarboxylic acid or their ester, lactone or salt. Suitable α-hydroxycarboxylic acids or α-ketocarboxylic acids are selected from lactic acid, tartaric acid, citric acid, 2-hydroxybutanoic acid, 2,3-dihydroxypropanoic acid, 2-hydroxypentanoic acid, 2-hydroxyhexanoic acid, 2-hydroxyheptanoic acid, 2-hydroxyoctanoic acid, 2-hydroxydecanoic acid, 2-hydroxydodecanoic acid, 2-hydroxytetradecanoic acid, 2-hydroxyhexadecanoic acid, 2-hydroxyoctadecanoic acid, mandelic acid, 4-hydroxymandelic acid, malic acid, meso-tartaric acid, tartaric acid, glucaric acid, galactaric acid, aldaric acid, gularic acid, 2-hydroxy-2-methylsuccinic acid, gluconic acid, pyruvic acid, glucuronic acid and galacturonic acid. The esters of the cited acids are selected from the methyl, ethyl, propyl, isopropyl, butyl, amyl, pentyl, hexyl, 2-ethylhexyl, octyl, decyl, dodecyl and hexadecyl esters. The α-hydroxycarboxylic acids or α-ketocarboxylic acids or their derivatives are comprised in amounts of 0.1 to 10 wt. %, preferably 0.5 to 5 wt. %, each based on the total composition.

Exemplary additional active substances, adjuvants and additives, which can be comprised in the inventive agents, are:
Vitamins, provitamins and vitamin precursors from the groups A, C, E and F, particularly 3,4-didehydroretinol (vitamin A2), β-carotene (provitamin of vitamin A1), ascorbic acid (vitamin C), as well as the esters of palmitic acid, glucosides or phosphates of ascorbic acid, tocopherols, particularly α-tocopherol as well as its ester, e.g. the acetate, the nicotinate, the phosphate and the succinate; in addition, vitamin F, under which are understood to mean essential fatty acids, particularly 1 inoleic acid, linolenic acid and arachidonic acid;
Allantoin,
Bisabolol,
Antioxidants, for example imidazoles (e.g. urocanic acid) and their derivatives, peptides such as D,L-carnosine, D-carnosine, L-carnosine and their derivatives (e.g. anserine), chlorogenic acid and their derivatives, liponic acid and their derivatives (e.g. dihydroliponic acid), aurothioglucose, propylthiouracil and other thiols (e.g. thioredoxine, glutathione, cystein, cystine, cystamine and their glycosyl-, n-acetyl-, methyl-, ethyl-, propyl-, amyl-, butyl- and lauryl-, palmitoyl-, oleyl-, γ-linoleyl-, cholesteryl- and glyceryl esters) as well as their salts, dilauryl thiodipropionate, distearyl thiodipropionate, thiodipropionic acid and derivatives thereof (esters, ethers, peptides, lipids, nucleotides, nucleosides and salts) as well as sulfoximine compounds (e.g. buthioninesulfoximines, homocysteine sulfoximine, butionine sulfone, penta-, hexa-, heptathionine sulfoximine) in very minor compatible doses (e.g. pmol to μmol/kg), further (metal)-chelates (e.g. α-hydroxyfatty acids, palmitic acid, phytinic acid, lactoferrin), humic acid, gallic acid, gall extracts, bilirubin, biliverdin, EDTA, EGTA and derivatives thereof, unsaturated fatty acids and their derivatives (e.g. γ-linolenic acid, linoleic acid, oleic acid), folic acid and derivatives thereof, ubiquinone and ubiquinol and their derivatives, coniferyl benzoate of benzoic resin, rutinic acid and derivatives thereof, α-glycosylrutine, ferula acid, furfurylidene glucitol, carnosine, butylhydroxytoluene, butylhydroxyanisole, nordihydroguajac resin acid, nordihydroguajaret acid, trihydroxybutyrophenone, uric acid and derivatives t hereof, catalases, superoxide-dismutase, zinc und its derivatives (e.g. ZnO, ZnSO₄) selenium and its derivatives (e.g. selenium-methionine), stilbenes and their derivatives (e.g. stilbene oxide, trans-stilbene oxide) and the derivatives suitable as antioxidants (salts, esters, ethers, sugars, nucleotides, nucleosides, peptides and lipids) of these active substances.
Ceramides and pseudoceramides,
Triterpenes, particularly triterpene acids such as ursolic acid, rosmarinic acid, betulinic acid, boswellic acid and bryonolic acid,
monomeric catechins, particularly catechin and epicatechin, leucoanthocyanidines, catechin polymers (catechin-tannins) as well as gallotannins,
Thickeners, e.g. gelatin, plant gums like agar-agar, guar gum, alginates, xanthane gum, gum arabica, karaya gum, locust bean flour, natural and synthetic clays such as e.g. bentonite, hectorite, montmorillonite or Laponite®, totally synthetic hydrocolloids such as e.g. polyvinyl alcohol, and also Ca—, Mg— or Zn salts of fatty acids,
plant glycosides,
structurants such as maleic acid and lactic acid,
dimethyl isosorbite,
alpha-, beta- as well as gamma cyclodextrines, particularly for stabilizing retinol,
solvents, swelling and penetration agents such as ethanol, isopropanol, ethylene glycol, propylene glycol, propylene glycol monoethyl ether, glycerin and diethylene glycol, carbonates hydrogen carbonates, guanidines, ureas as well as primary, secondary and tertiary phosphates
perfume oils, pigments as well as colorants to dye the agent,
pH adjusters, e.g. α- and β-hydroxycarboxylic acids,
chelating agents like EDTA, NTA, β-alanine diacetic acid and phosphonic acids,
opacifiers like latex, styrene/PVP copolymers and styrene/acrylamide copolymers,
pearlizing agents like ethylene glycol mono and distearate as well as PEG-3-distearate,
blowing agents like propane-butane mixtures, N₂O, dimethyl ether, CO₂ and air,

In a preferred embodiment, the inventive skin treatment agents are present in the form of liquid or solid oil-in-water emulsions, water-in-oil emulsions, multiple emulsions, micro-emulsions, PIT-emulsions or Pickering emulsions, a hydrogel, a lipogel, a mono or multiphase solution, a foam, a powder or a mixture with at least one polymer that is a suitable medicinal adhesive. The agents can also be presented in an anhydrous state, such as for example in an oil or a balsam. For this, the carrier can be a vegetal or animal oil, a mineral oil, a synthetic oil or a mixture of such oils.

In a particular embodiment of the inventive agents, the agents are present as a microemulsion. In the context of the invention, microemulsions are understood to mean both thermodynamically stable microemulsions as well as the so-called “PIT” emulsions. These emulsions are a system with the three components water, oil and emulsifier, which are present at room temperature as oil-in-water emulsions. On heating, these systems form microemulsions in a specific temperature range (known as the phase inversion temperature or “PIT”) and on further heating are converted into water-in-oil (W/O) emulsions. Subsequent cooling again affords O/W emulsions that also exist, however, at room temperature as microemulsions or as finely dispersed emulsions with a mean particle diameter below 400 nm and particularly between about 100-300 nm. According to the invention, such micro or “PIT” emulsions that have a mean particle diameter of about 200 nm are preferred. Details of “PIT emulsions” are to be found for example in the publication Angew. Chem. 97, 655-669 (1985).

Anti-perspirant active principles can also be included in the inventive composition. According to the invention, water-soluble astringent or protein-coagulating metallic salts are suitable as the active principles for antiperspirants, particularly inorganic or organic salts of aluminum, zirconium, zinc and titanium as well as any mixtures of these salts. According to the invention, water-solubility is understood to mean a solubility of at least 4 g of active substance per 100 g of solution at 20° C. Exemplary agents according to the invention include alum (KA1(SO₄)₂.12H₂O), aluminum sulfate, aluminum lactate, sodium-aluminum chloro hydroxy lactate, aluminum chloro hydroxy allantoinate, aluminum chlorohydrate, aluminum sulfocarbolate, aluminum-zirconium chlorohydrate, zinc chloride, zinc sulfocarbolate, zinc sulfate, zirconium chlorohydrate, aluminum-zirconium-chlorohydrate-glycine-complexes and complexes of basic aluminum chlorides with propylene glycol or polyethylene glycol. The liquid preparations of active principles preferably comprise an astringent aluminum salt, particularly aluminum chlorohydrate, and/or an aluminum-zirconium compound. Aluminum chlorohydrates are commercialized, for example, in powder form as Micro Dry® Ultrafine or in activated form as Reach® 501 or Reach® 103 from Reheis as well as in the form of aqueous solutions as Locron® L from Clariant or as Chlorhydrol® from Reheis. An aluminum sesquichlorohydrate is offered by Reheis under the trade name Reach® 301. Also, the use of aluminum-zirconium tri or tetrachlorohydrex-glycine complexes, which are commercialized, for example by Reheis under the trade name Rezal® 36G, is particularly advantageous according to the invention.

The perspiration-inhibiting active principle is comprised in the inventive compositions in an amount of 0.01 to 40 wt. %, preferably 2 to 30 wt. % and particularly 5 to 25 wt. %, based on the amount of the active substance in the total composition.

Deodorant active principles can also be included in the compositions described herein. According to the invention, fragrances, antimicrobials, antibacterials or germ-inhibitors, enzyme-inhibitors, antioxidants and odor adsorbents are suitable deodorant active principles.

Suitable antimicrobial, antibacterial or germ-inhibiting materials are particularly C₁-C₄ alkanols, C₂-C₄ alkane diols, organohalogen compounds as well as organo halides, quaternary ammonium compounds, a series of plant extracts and zinc compounds.

In a further preferred embodiment, the inventive compositions comprise at least one water-soluble polyol, selected from water-soluble diols, triols and higher polyhydroxy alcohols as well as polyethylene glycols. Suitable diols include C₂-C₁₂-diols, particularly 1,2-propylene glycol, butylene glycols such as e.g. 1,2-butylene glycol, 1,3-butylene glycol and 1,4-butylene glycol, pentanediols, e.g. pentane-1,2-diol, as well as hexanediols, e.g. hexane-1,6-diol. In addition, glycerin and technical oligoglycerin mixtures with a self-condensation degree of 1.5 to 10 such as technical diglycerin mixtures with a diglycerin content of 40 to 50 wt. % or triglycerin, are preferably suitable, further hexane-1,2,6-triol as well as polyethylene glycol (PEG) with an average molecular weight of 100 to 1000 daltons, for example PEG-400, PEG-600 or PEG-1000. Additional suitable higher polyhydroxy alcohols are the C₄-, C₅- and C₆-monosaccharides and the corresponding sugar alcohols, e.g. mannitol or sorbitol.

The inventive compositions comprise the water-soluble polyol in amounts of 1-50 wt. %, preferably 1-15 wt. % and particularly preferably 1-5 wt. %, in each case based on the total composition.

The following examples are intended to clarify the invention without limiting it in any way.

### EXAMPLE 1

### Influence of Plant Extracts on the Growth of Staphylococcus Warneri and Propionibacterium Acnes

Cultures in LB-medium with an optical density OD (620 nm) of 0.05 were seeded from liquid starter cultures of S. warneri and P. Acnes. In parallel to the controls (without the addition of cells or extract), 2 cultures were each spiked with 1% plant extract and the growth documented over a 30-hour period by means of OD measurements. After 30 hours the difference in OD of the cultures with added extract to the corresponding controls (without added extract, adjusted for the value without cells) was determined. A mixed extract of Ribes nigrum and Pinus sylvestris (Epica®, Rahn (Deutschland)) as well as extracts of Picea glauca, Paullinia cupana (Guarana), Panax ginseng (Ginseng), Lamium album (white nettle) and Ribes nigrum (blackcurrant) had a growth-promoting effect on S. warneri with concomitant inhibition of P. acnes. The extracts were obtained from the Rahn Company (Germany).

TABLE 1
Growth effect of various plant extracts on S. warneri and P. acnes
(OD differences from the control after 30 hours)

| | | Ribes | | | | |
|---|---|---|---|---|---|---|
| | | nigrum/ | | | | |
| | | Pinus | | White | Ribes | Picea |
| | Guarana | sylvestris | Ginseng | Nettle | nigrum/ | glauca |
| S. warneri | 0.41 | 0.22 | 0.48 | 0.01 | 0.13 | 0.04 |
| P. acnes | −0.04 | −0.07 | −0.02 | −0.001 | −0.02 | −0.05 |

### Formulation Examples

The data each refer to the content in wt. %.

| | Lamellar Cream. O/W-emulsion |
|---|---|
| Ingredient | [wt. %] |
| Cetiol ® OE | 7.0 |
| Cetiol ® V | 7.0 |
| Lanette ® 22 | 7.0 |
| Lanette ® E | 0.18 |
| Baysilonöl M 350 | 0.5 |
| Vitamin E-acetate | 1.0 |
| Retinyl palmitate | 1.0 |
| D-Panthenol | 1.0 |
| Prebiotic Plant extract | 1.0 |
| Glycerin | 5.0 |
| Formalin solution (37%) | 0.08 |
| Water | ad 100 |

| | Example 3.1: | |
|---|---|---|
| | O/W-PIT- | Example 3.2: |
| | Emulsion | W/O-Emulsion |
| Ingredient | [wt. %] | [wt. %] |
| Cetiol ® OE | 7.5 | 7.0 |
| Cetiol ® V | 7.5 | 7.0 |
| Lanette ® O | 4.0 | — |
| Glyceryl palmitate | 2.2 | — |
| Eumulgin ® B 2 | 2.1 | — |
| Baysilonöl M 350 | 0.5 | — |
| Vitamin E-acetate | 1.0 | — |
| Retinyl palmitate | 1.0 | 1.0 |
| Biotin | 0.005 | — |
| Dihydro-3-hydroxy-4.4-dimethyl- | 1.0 | 1.0 |
| 2(3H)-furanone (Pantolactone) | | |
| Algae extract SPHM 3002 | — | 1.0 |
| Prebiotic Plant extract | 1.0 | 1.0 |
| Glycerin | 5.0 | 5.0 |
| MgSO₄.H₂O | — | 0.7 |
| Formalin solution (37%) | 0.08 | 0.08 |
| Water | ad 100 | ad 100 |

| | Example 3.3: | | |
|---|---|---|---|
| | Lipoprotein- | Example 3.4: | |
| Ingredient | Cream | Glycolipid-Cream | Example 3.5: |
| Montanov ® 202 | — | — | 4.0 |
| Thistle oil | 3.0 | — | — |
| Evening primrose oil | — | 3.0 | — |
| Myritol ® PC | 3.5 | 3.5 | — |
| Myritol ® 331 | — | — | 3.0 |
| Myritol ® 318 | — | — | 2.0 |
| Cetiol ® MM | — | 2.5 | — |
| Cetiol ® B | — | — | 7.0 |
| Cetiol ® SB 45 | — | — | 0.5 |
| Lanette ® 22 | 3.0 | — | — |
| Cutina ® GMS-V | 3.0 | 4.0 | 2.0 |
| Lanette ® O | 3.0 | 2.0 | 1.0 |
| Edenor ® IPS | 6.0 | 6.0 | — |
| Cosmacol ® PLG | — | 3.0 | — |
| Baysilonöl M350 | 1.0 | 1.0 | 0.5 |
| Eusolex ® 6300 | 0.6 | 0.6 | 3.0 |
| Parsol ® 1789 | 0.1 | 0.1 | 2.0 |
| Controx ® KS | 0.05 | 0.05 | 0.05 |
| pHB-Propyl ester | 0.2 | — | 0.2 |
| Prebiot. plant extract | 1.0 | 1.0 | 1.0 |
| Panthenol | 1.0 | 1.0 | 1.0 |
| Herbasol ® distillate | — | 1.0 | — |
| Mallow | | | |
| Herbasol ® extract | — | — | 1.0 |
| Rosemary | | | |
| Dry Flo ® Plus | — | 3.0 | — |
| 1.6-Hexanediol | — | 6.0 | — |
| Dipropylene glycol | — | 5.0 | — |
| Glycerin | 5.0 | — | — |
| DSC-H N | — | 5.0 | — |
| V-Protein liquid | 9.0 | — | — |
| Tioveil ®-AQ-N | 2.0 | — | — |
| Citric acid | 0.1 | — | — |
| Sepigel ® 305 | 3.0 | 0.4 | — |
| Methocel ® E 4M | — | — | 0.2 |
| Herbasol ® distillate of | — | 1.0 | — |
| Green Tea | | | |
| water | ad 100 | ad 100 | ad 100 |

| | Example 3.6: |
|---|---|
| Ingredient | Mild cleaning gel |
| Eumulgin ® HRE 40 | 0.6 |
| Eucarol ® AGE-ET | 2.0 |
| 1.2-Propylene glycol | 10.0 |
| Prebiotic Plant extract | 1.0 |
| Bisabolol | 0.1 |
| D-Panthenol | 0.5 |
| pHB-Propyl ester | 0.1 |
| pHB-Methyl ester | 0.2 |
| Carbopol ® ETD 2020 (0.5%) | 50.0 |
| Water | ad 100 |

| | Example 3.7 | Example 3.8 |
|---|---|---|
| Ingredient | Matrix plaster: | Matrix plaster: |
| DURO-TAK ® | 76 | 76 |
| Prebiotic Plant extract | 1.0 | 1.0 |
| Ultrasome ™ | — | 0.1 |
| Panthenol | 2 | — |
| Dihydro-3-hydroxy-4.4-dimethyl- | — | 2 |
| 2(3H)-furanone (Pantolactone) | | |
| Herbasol ® Distillate of | 1 | — |
| marshmallow | | |
| Herbasol ® distillate of Green Tea | — | 1 |
| Aloe Vera Gel | 1 | 1 |
| Tioveil ®-AQ-N | 2 | — |
| Eusolex ® OCR | 1 | — |
| Propylene glycol monooleate | 5 | 5 |
| Controx ® KS | 0.05 | 0.05 |
| Water | ad 100 | ad 100 |

| | Example 3.9 | Example 3.10 |
|---|---|---|
| | Reservoir | Reservoir |
| Ingredients of the active reservoir | plaster: | plaster: |
| Prebiotic Plant extract | 1.0 | 1.0 |
| Ultrasome ™ | — | 0.1 |
| Panthenol | 1.0 | — |
| Pantolactone | | 1.0 |
| Bisabolol | 1.0 | 1.0 |
| Herbasol ® Distillate of marshmallow | — | 1.0 |
| Ethanol | 40 | 40 |
| Mowiol ® 18-88 | 8.0 | 8.0 |
| Luviskol ® K 80 | 5.0 | 5.0 |
| Controx ® KS | 0.05 | 0.05 |
| Brij ®-35 | 2.0 | 2.0 |
| Cremophor ® CO-40 | 0.5 | 0.5 |
| Glycerin | 5.0 | 5.0 |
| Water | ad 100 | ad 100 |

4. Oil in Water Emulsions

4.1. Example Series:

| | 1 | 2 | 3 |
|---|---|---|---|
| Carthamus Tinctorius | 3.00 | 3.00 | 3.00 |
| Caprylic/Capric Triglyceride | 5.00 | 5.00 | 5.00 |
| Coco glycerides | 2.00 | 2.00 | 2.00 |
| Behenyl Alcohol | 1.00 | 1.00 | 1.00 |
| Glyceryl Stearate | 2.00 | 2.00 | 2.00 |
| Cetearyl Alcohol | 1.00 | 1.00 | 1.00 |
| Isopropyl stearate | 4.00 | 4.00 | 4.00 |
| Shea Butter | 2.00 | 2.00 | 2.00 |
| Dimethicone | 1.00 | 1.00 | 1.00 |
| Hydrogenated Palm Glycerides | 0.05 | 0.05 | 0.05 |
| Citrate | | | |
| Propyl paraben | 0.20 | 0.20 | 0.20 |
| Cyclopentasiloxane/Dimethiconol | 1.00 | 1.00 | 1.00 |
| Aluminum Starch Octenyl succinate | 1.00 | 1.00 | 1.00 |
| TiO2 | 0.50 | 0.50 | 0.50 |
| Hexanediol | 6.00 | 3.00 | |
| Propylene glycol | 5.00 | 5.00 | 5.00 |
| Glycerin | 5.00 | 3.00 | 3.00 |
| Methylparaben | 0.20 | 0.20 | 0.20 |
| Sodium Carbomer | 0.40 | 0.40 | 0.40 |
| Dimethylsilanol Hyaluronate | 5.00 | 5.00 | 5.00 |
| Algae Extract | 1.00 | | |
| Prebiotic Plant extract | 0.5 | 0.8 | 1.2 |
| Perfume | 0.10 | 0.10 | 0.10 |
| Aqua | ad. 100 | ad. 100 | ad. 100 |

4.2. Example Series:

| | 1 | 2 | 3 |
|---|---|---|---|
| Cetearyl Isononanoate | 4.00 | 4.00 | 4.00 |
| Mineral oil | 6.00 | 6.00 | 6.00 |
| Glyceryl Stearate/Cetearyl Alcohol/ | 2.00 | 2.00 | 2.00 |
| Cetyl Palmitate/Cocoglycerides | | | |
| Palmitic Acid/Stearic Acid | 1.50 | 1.50 | 1.50 |
| Ceteareth-30 | 1.00 | 1.00 | 1.00 |
| Dimethicone | 1.00 | 1.00 | 1.00 |
| Tocoperyl Acetate | 0.50 | 0.50 | 0.50 |
| Propyl paraben | 0.30 | 0.30 | 0.30 |
| Sweet Almond Oil | 2.00 | 2.00 | 2.00 |
| Acrylates/C10-30 Alkyl Acrylate | 0.27 | 0.27 | 0.27 |
| Crosspolymer | | | |
| Glycerin | 5.00 | 3.00 | 3.00 |
| Lactic Acid | 0.26 | 0.26 | 0.26 |
| Propylene glycol | 5.00 | 5.00 | 5.00 |
| Methylparaben | 0.30 | 0.30 | 0.30 |
| Phenoxyethanol | 0.90 | 0.90 | 0.90 |
| Panthenol | 0.50 | | |
| Laminaria Digitata Extract | 1.00 | | |
| Sodium Chloride | 0.05 | 0.05 | 0.05 |
| Polyacrylamide/C13-14 | 0.50 | 0.50 | 0.50 |
| Isoparaffin/Laureth-7 | | | |
| Silk Protein | 0.25 | 0.25 | 0.25 |
| Xanthane Gum | 0.20 | 0.20 | 0.20 |
| Prebiotic Plant extract | 0.6 | 0.8 | 1.0 |
| Perfume | 0.30 | 0.30 | 0.30 |
| Aqua | ad. 100 | ad. 100 | ad. 100 |

4.3. Example Series:

| | 1 | 2 | 3 |
|---|---|---|---|
| Hydrogenated Lecithin | 0.50 | 0.50 | 0.50 |
| Isopropyl stearate | 4.00 | 4.00 | 4.00 |
| Dibutyl Adipate | 2.00 | 2.00 | 2.00 |
| Tocopheryl Acetate | 0.50 | 0.50 | 0.50 |
| Glyceryl Stearate | 1.00 | 1.00 | 1.00 |
| Behenyl Alcohol | 2.00 | 2.00 | 2.00 |
| Dimethicone | 0.50 | 0.50 | 0.50 |
| Propyl paraben | 0.20 | 0.20 | 0.20 |
| Cyclomethicone/Dimethicone | 1.00 | 1.00 | 1.00 |
| Crosspolymer | | | |
| Glycerin | 4.50 | 3.00 | 3.00 |
| Hexanediol | 6.00 | 3.00 | |
| Methylparaben | 0.20 | 0.20 | 0.20 |
| Sodium Carbomer | 0.30 | 0.30 | 0.30 |
| Dimethyl Silanol Hyaluronate | 5.00 | 5.00 | 5.00 |
| Algae Extract | 1.00 | | |
| Plankton Extract | 0.20 | 0.20 | 0.20 |
| Dimethylmethoxy Chromanol | 0.01 | 0.01 | 0.01 |
| Propylene glycol | 5.00 | 5.00 | 5.00 |
| Palmitoyl Pentapeptide-3 | 3.00 | | |
| Palmitoyl Oligopeptide | | 2.00 | |
| Palmitoyl Tetrapeptide-1 | | 1.00 | |
| Hydroxyethyl Acrylate/Sodium | 1.50 | 1.50 | 1.50 |
| Acryloyldimethyl Taurate | | | |
| Copolymer/Squalane/Polysorbate | | | |
| 60 | | | |
| TiO₂ | 0.50 | 0.50 | |
| Prebiotic Plant extract | 0.5 | 0.9 | 1.3 |
| Perfume | 0.35 | 0.35 | 0.35 |
| Aqua | ad. 100 | ad. 100 | ad. 100 |

4.4. Example Series:

| | 1 | 2 | 3 | 4 | 5 |
|---|---|---|---|---|---|
| Cetearyl Alcohol/Cetearyl | 5.00 | 5.00 | 5.00 | 5.00 | 5.00 |
| Glucoside | | | | | |
| Caprylic/Capric Triglyceride | 5.00 | 5.00 | 5.00 | 5.00 | 5.00 |
| Shea Butter | 0.50 | 0.50 | 0.50 | 0.50 | 0.50 |
| Coco glycerides | 2.00 | 2.00 | 2.00 | 2.00 | 2.00 |
| Cetearyl Alcohol | 1.00 | 1.00 | 1.00 | 1.00 | 1.00 |
| Glyceryl Stearate | 2.00 | 2.00 | 2.00 | 2.00 | 2.00 |
| Dimethicone | 0.50 | 0.50 | 0.50 | 0.50 | 0.50 |
| Tocoperyl Acetate | 0.50 | 0.50 | 0.50 | 0.50 | 0.50 |
| Hydrogenated Palm Glycerides Citrate | 0.25 | 0.25 | 0.25 | 0.25 | 0.25 |
| Polysilicone-15 | | 4.00 | 4.00 | | |
| Phenylbenzimidazole Sulfonic | | 2.00 | 2.00 | 2.00 | |
| Acid | | | | | |
| Disodium Phenyl | | | | 1.00 | |
| Dibenzimidazole Tetrasulfonate | | | | | |
| Octocrylene | | | | 5.00 | |
| 4-Methoxybenzylidene Camphor | 2.00 | | | | |
| Butyl- | 1.00 | 1.80 | 1.80 | | |
| methoxydibenzoylmethane | | | | | |
| Propyl paraben | 0.20 | 0.20 | 0.20 | 0.20 | 0.20 |
| Sodium Carbomer | 0.50 | 0.50 | 0.50 | 0.50 | 0.50 |
| Hexanediol | 6.00 | 6.00 | 3.00 | 6.00 | |
| Talc | 0.50 | 0.50 | 0.50 | 0.50 | 0.50 |
| Methylparaben | 0.20 | 0.20 | 0.20 | 0.20 | 0.20 |
| Glycerin | 4.50 | 4.50 | 3.00 | 4.50 | 3.00 |
| Aluminum Starch Octenyl | 1.00 | 1.00 | 1.00 | 1.00 | 1.00 |
| succinate | | | | | |
| Hydrogenated Lecithin | 1.00 | 1.00 | 1.00 | 1.00 | 1.00 |
| Fagus Silvatica Extract | 2.00 | 2.00 | 2.00 | 2.00 | 2.00 |
| Hydrolyzed Soy Protein | 2.00 | 2.00 | 2.00 | 2.00 | 2.00 |
| Dimethylsilanol Hyaluronate | 2.00 | 2.00 | 2.00 | 2.00 | 2.00 |
| Trisodium NTA | 0.10 | 0.10 | 0.10 | 0.10 | 0.10 |
| Phenoxyethanol | 0.40 | 0.40 | 0.40 | 0.40 | 0.40 |
| Hydrolyzed Wheat Protein | 3.00 | 3.00 | 3.00 | 3.00 | 3.00 |
| Prebiotic Plant extract | 0.8 | 1.2 | 1.0 | 1.0 | 1.2 |
| Perfume | 0.40 | 0.40 | 0.40 | 0.40 | 0.40 |
| Aqua | ad. 100 | ad. 100 | ad. 100 | ad. 100 | ad. 100 |

4.5. Example Series:

| | 1 | 2 | 3 |
|---|---|---|---|
| C14-22 Alcohols/C12-20 Alkyl | 3.00 | 3.00 | 3.00 |
| Glucoside | | | |
| Dibutyl Adipate | 6.00 | 6.00 | 6.00 |
| Caprylic/Capric Triglyceride | 3.00 | 3.00 | 3.00 |
| Cetearyl Alcohol | 1.00 | 1.00 | 1.00 |
| Glyceryl Stearate | 0.50 | 0.50 | 0.50 |
| Dimethicone | 0.50 | 0.50 | 0.50 |
| Propyl paraben | 0.20 | 0.20 | 0.20 |
| Cyclopentasiloxane/Dimethiconol | 1.50 | 1.50 | 1.50 |
| Glycerin | 5.00 | 5.00 | 5.00 |
| Sorbitol | 3.00 | 3.00 | 3.00 |
| Methylparaben | 0.20 | 0.20 | 0.20 |
| Aluminum Starch Octenyl succinate | 0.50 | 0.50 | 0.50 |
| Sodium Carbomer | 0.10 | 0.10 | 0.10 |
| Dimethylsilanol Hyaluronate | 2.00 | 2.00 | 2.00 |
| Laminata Digitata Extract | 0.50 | 0.50 | 0.50 |
| Hydroxyethyl Acrylate/Sodium | 1.00 | 1.00 | 1.00 |
| Acryloyldimethyl Taurate | | | |
| Copolymer/Squalane/Polysorbate | | | |
| 60 | | | |
| Prebiotic Plant extract | 0.4 | 0.8 | 1.2 |
| Perfume | 0.20 | 0.20 | 0.20 |
| Aqua | ad. 100 | ad. 100 | ad. 100 |

5. Water-in-oil Emulsion

| | | |
|---|---|---|
| 1 | 2 | 3 |

| | | | |
|---|---|---|---|
| Polyglyceryl-3 Diisostearate | 3.0 | 3.0 | 3.0 |
| PEG-45/Dodecyl Glycol Copolymer | 0.5 | 0.5 | 0.5 |
| Microcrystalline Wax | 3.0 | 3.0 | 3.0 |
| Bis-Diglyceryl Polyacyl adipate-2 | 1.0 | 1.0 | 1.0 |
| Paraffin oil | 8.0 | 8.0 | 8.0 |
| Vaseline | 2.0 | 2.0 | 2.0 |
| Vitamin-E-acetate | 2.0 | 2.0 | 2.0 |
| Methylparaben | 0.3 | 0.3 | 0.3 |
| Propyl paraben | 0.3 | 0.3 | 0.3 |
| Isopropyl isostearate | 8.0 | 8.0 | 8.0 |
| Glycerin | 5.0 | 3.0 | 3.0 |
| Mg-Sulfate | 0.5 | 0.5 | 0.5 |
| plant extract | 0.8 | 1.2 | 1.5 |
| Lactic acid 80% | 0.560 | 0.560 | 0.560 |
| Algae Extract | 1.0 | | |
| Panthenol | 0.5 | 1.0 | 0.5 |
| Calendula Officinalis Flower Extract | 0.3 | | |
| Propylene Glycol | 0.5 | | |
| Perfume | 0.2 | 0.2 | 0.2 |
| Aqua | ad.100 | ad.100 | ad.100 |

6. Cleaning Preparations

6.1. Example Series:

| | 1 | 2 | 3 |
|---|---|---|---|
| Dipropylene glycol | 10.00 | 10.00 | 10.00 |
| Chlorhexidine digluconate | 1.00 | 1.00 | 1.00 |
| Poloxamer-184 | 3.00 | 3.00 | 3.00 |
| Panthenol | 0.50 | 0.50 | 0.50 |
| Chitosan Glycolate | 3.00 | 3.00 | 3.00 |
| PEG-40 Hydrogenated Castor Oil/ | 0.50 | 0.50 | 0.50 |
| Trideceth 9/Propylene Glycol | | | |
| Chlorella Vulgaris Extract | 0.50 | 0.50 | 0.50 |
| Prebiotic Plant extract | 0.4 | 0.8 | 1.2 |
| Perfume | 0.20 | 0.20 | 0.20 |
| Aqua | ad. 100 | ad. 100 | ad. 100 |

6.2. Example Series:

| | 1 | 2 | 3 |
|---|---|---|---|
| Carbomer | 1.40 | 1.40 | 1.40 |
| Sorbitol | 2.10 | 2.10 | 2.10 |
| Sodium Benzoate | 0.40 | 0.40 | 0.40 |
| Lauryl Glucoside | 7.50 | 7.50 | 7.50 |
| Cocoamidopropyl betaine | 3.40 | 3.40 | 3.40 |
| Disodium Laureth Sulfosuccinate | 5.00 | 5.00 | 5.00 |
| PEG-7 Glyceryl Cocoate | 0.50 | 0.50 | 0.50 |
| Coco-Glucoside/Glyceryl Oleate | 5.00 | 5.00 | 5.00 |
| Hydrogenated Palm Glycerides | 0.05 | 0.05 | 0.05 |
| Citrate | | | |
| Sodium PCA | 1.60 | 1.60 | |
| Pantolactone | 1.00 | 1.00 | |
| Tetrasodium EDTA | 0.25 | 0.25 | 0.25 |
| Sodium Lactate | 1.80 | | |
| Panthenol | 0.50 | 0.50 | 0.50 |
| Prebiotic Plant extract | 0.8 | 1.2 | 1.6 |
| Perfume | 0.40 | 0.40 | 0.40 |
| Aqua | ad. 100 | ad. 100 | ad. 100 |

7. Water-in-Silicone Emulsion

| | 1 | 2 | 3 |
|---|---|---|---|
| Dimethicone | 2.50 | 2.50 | 2.50 |
| Cyclomethicone | 25.00 | 25.00 | 25.00 |
| Cetyl Dimethicone | 2.00 | 2.00 | 2.00 |
| Copolyol | | | |
| Sodium Chloride | 2.00 | 2.00 | 2.00 |
| Perfume | 0.30 | 0.30 | 0.30 |
| Chlorhexidine digluconate | 0.20 | 0.20 | 0.20 |
| Prebiotic Plant extract | 0.4 | 0.8 | 1.2 |
| Aqua | ad. 100 | ad. 100 | ad. 100 |

Raw Materials Used:

| Name | INCI |
|---|---|
| Algae extrakt SPHM 3002 | Aqua, Algae (Linne) |
| Aloe Vera Gel (Provital SA): | Aloe Barbadensis (Linne) |
| 0.85-1.55 wt. % active substance in | |
| propylene glycol/water | |
| Baysilonöl M 350 | Polydimethylsiloxane/Dimethicone |
| Brij ®-35 | Laureth-23 |
| Carbopol ® ETD 2020 (0.5%) | Acrylates/C10-30 Alkyl Acrylate |
| | Crosspolymer |
| Eumulgin ® B 2 | Ceteareth-20 |
| Cetiol ® B | Dibutyl Adipate |
| Cetiol ® MM | Myristyl Myristate |
| Cetiol ® SB 45 | Butyrospermum Parkii (Linne) |
| Controx ® KS: | Tocopherol, Hydrogenated Palm |
| | Glycerides Citrate |
| Cosmacol ® PLG | Tri-C12-13 Alkyl Citrate |
| Cremophor ® CO-40 | PEG-40 Hydrogenated Castor Oil |
| Cutina ® GMS (C16-18-Fatty acid mono-di- | Glyceryl Stearate |
| glyceride | |
| Cetiol ® V | Decyl Oleate |
| Cetiol ® OE | Dicaprylyl ether |
| Dry Flo ® Plus | Aluminum Starch Octenyl succinate |
| DSC-H N (ex Exsymol) | Dimethylsilanol Hyaluronate |
| DURO-TAK ® (National Starch and | Polyacrylate Copolymer |
| Chemical): ca. 50% Acrylate-Copolymer in | |
| Benzine/Ethyl acetate/Methanol/Ethanol | |
| Eucarol ® AGE-ET UP (30% active | Sodium Cocopolyglucose Tartrate |
| substance in water) | |
| Eumulgin ® HRE 40 | PEG-40 Hydrogenated Castor Oil |
| Eusolex ® 6300 | 4-Methylbenzylidene Camphor |
| Eusolex ® OCR: | Octocrylene |
| Herbasol ® Distillate of marshmallow | Water, Alcohol denat., Althea officinalis |
| (Cosmetochem) | |
| Herbasol ® distillate of Green Tea | Water, Camellia sinensis extract |
| Herbasol ® distillate mallow (Cosmetochem) | Aqua, SD Alcohol 39-C, Malva |
| | Sylvestris (Linne) |
| Herbasol ® Extract Rosemary | Water, Propylene Glycol, Rosmarinus |
| | officinalis |
| Edenor ® IPS | Isopropyl Stearate |
| Lanette ® E | Sodium Cetearyl Sulfate |
| Lanette ® O | Cetearyl Alcohol |
| Lanette ® 22 | Behenyl Alcohol |
| Lifidrem ® PPST-GHK-4 (Coletica): Pea | Pea Extract (Pisum Sativum (Linne)), |
| protein-Extract/C₁₆₋₁₈ fatty acid condensate | Sodium Stearate, Sodium Chloride |
| Methocel ® E 4M | Hydroxypropyl Methylcellulose |
| Montanov ® 202 | Arachidyl Alcohol, Behenyl Alcohol, |
| | Arachidyl Glucoside |
| Myritol ® 318 | Caprylic/Capric Triglyceride |
| Myritol ® 331 | Coco glycerides |
| Myritol ® PC | Propylene Glycol Dicaprylate/Dicaprate |
| Evening primrose oil | Evening Primrose Oil Oenothera Biennis |
| | (Linne) |
| Parsol ® 1789 | Butyl Methoxydibenzoylmethane |
| pHB-Propyl ester | Propyl paraben |
| Photosome ™ | Plankton Extract and Lecithin |
| Mowiol ® 18-88 | Polyvinyl alcohol, partially saponified |
| Luviskol ® K 80 | polyvinyl pyrrolidone |
| Sepigel ® 305 | Polyacrylamide, C13-14 Isoparaffin, |
| | Laureth-7 |
| Tioveil ®-AQ-N (Uniqema): Titanium | CI 77891 (Titanium Dioxide), Alumina, |
| dioxide dispersion | Silica, Sodium Polyacrylate |
| Ultrasome ™ | Micrococcus lysate |
| V-Protein liquid COS 152/22 A | Aqua, Propylene Glycol, Hydrolyzed Pea |
| (Cosmetochem) | Protein (Pisum Sativum) |
| Vitamin E-acetate | Tocopheryl Acetate |

What is claimed is:

1. A composition comprising at least one plant extract that has a prebiotic effect on the skin in an acceptable carrier.

2. The composition according to claim 1, wherein said plant extract comprises a topical skin treatment agent, wherein said plant extract is present in 0.01 to 20 wt % based of the total weight of the composition.

3. The composition according to claim 2, wherein the plant extract is selected from the group consisting of at least one of a conifer extract, a Pinacae extract, a Sapindaceae extract, an Araliaceae extract, a Lamiaceae extract and a Saxifragaceae extract.

4. The composition according to claim 3, wherein the plant extract comprises an extract of at least one of Picea spp., Pinus sp., Paullinia sp., Panax sp., Lamium sp. and Ribes sp.

5. The composition according to claim 3, wherein said composition comprises at least one additional substance selected from the group consisting of organic light protecting filters, mineral light protecting filters, modified mineral light protecting filters, vitamins, provitamins, vitamin precursors of the vitamin B group or their derivatives, derivatives of 2-furanone, panthenol, pantolactone, nicotinamide, biotin, MMP-1-inhibiting substances, and esters of retinol (vitamin A1) with a C₂₋₁₈ carboxylic acid.

6. The composition according to claim 1, wherein said composition comprises at least one additional substance, selected from the group consisting of surface active substances, amino acids, amino acid zinc salts, amino acid addition salts, film-forming polymers, emulsion-stabilizing polymers, thickening polymers, adhesive polymers, fats, surfactants, anti-perspirants and polyols, protein hydrolyzates, protein hydrolyzate derivatives, monosaccharides, oligosaccharides, polysaccharides, saccharide derivatives, α-hydroxycarboxylic acids, α-ketocarboxylic acids α-hydroxycarboxylic acid esters, α-hydroxycarboxylic acid lactones, salts of α-hydroxycarboxylic acid, α-ketocarboxylic acid esters, α-ketocarboxylic lactones, and salts of α-ketocarboxylic acid.

7. The composition according to claim 5, wherein said composition comprises at least one additional substance, selected from the group consisting of surface active substances, amino acids, amino acid zinc salts, amino acid addition salts, film-forming polymers, emulsion-stabilizing polymers, thickening polymers, adhesive polymers, fats, surfactants, anti-perspirants and polyols, protein hydrolyzates, protein hydrolyzate derivatives, monosaccharides, oligosaccharides, polysaccharides, saccharide derivatives, α-hydroxycarboxylic acids, α-ketocarboxylic acids α-hydroxycarboxylic acid esters, α-hydroxycarboxylic acid lactones, salts of α-hydroxycarboxylic acid, α-ketocarboxylic acid esters, α-ketocarboxylic lactones, and salts of α-ketocarboxylic acid.

8. The composition according to claim 1, wherein said composition is comprised in a soap, a lotion, an emulsion, a spray, a cream, a shampoo and a plaster.

9. The composition according to claim 5, wherein said composition is comprised in a soap, a lotion, an emulsion, a spray, a cream, a shampoo and a plaster.

10. The composition according to claim 6, wherein said composition is comprised in a soap, a lotion, an emulsion, a spray, a cream, a shampoo and a plaster.

11. The composition according to claim 7, wherein said composition is comprised in a soap, a lotion, an emulsion, a spray, a cream, a shampoo, and a plaster.

12. A skin treatment method for modulating the growth of skin microbes comprising administration of an effective amount of the composition according to claim 1.

13. The method of claim 12, wherein the growth of desirable saprophytic skin microbes is promoted.

14. The method according to claim 12, wherein the growth of desirable coagulase-negative staphylococci is promoted.

15. The method of claim 14, wherein the coagulase-negative staphylococci are selected from the group consisting of S. epidermidis or S. warneri.

16. The method of claim 12, wherein the growth of desirable bacilli is promoted.

17. The method according to claim 16, wherein the bacilli are Bacillus licheniformis.

18. The method of claim 12, wherein said plant extracts having prebiotic effects inhibit the growth of undesirable skin microbes.

19. The method of claim 18, wherein the microbes are pathogenic microbes.

20. The method of claim 19, wherein the microbes are coagulase-positive staphylococci.

21. The method of claim 19, wherein the microbes are Propionibacterium acnes.

22. The method of claim 12, wherein said composition is effective to treat bad, dry or greasy skin.

23. The method of claim 12, wherein said composition is effective to treat acne.

24. The method of claim 12, wherein said composition is effective to treat skin fungi or dandruff.

25. The method of claim 12, wherein said composition comprises a plant extract selected from the group consisting of at least one of a conifer extract, a Pinacae extract, a Sapindaceae extract, an Araliaceae extract, a Lamiaceae extract and a Saxifragaceae extract.

26. The method of claim 25, wherein the plant extract comprises an extract of at least one of Picea spp., Pinus sp., Paullinia sp., Panax sp., Lamium sp. and Ribes sp.

27. The method of claim 25, wherein said composition comprises at least one additional substance selected from the group consisting of organic light protecting filters, mineral light protecting filters, modified mineral light protecting filters, vitamins, provitamins, vitamin precursors of the vitamin B group or their derivatives, derivatives of 2-furanone, panthenol, pantolactone, nicotinamide, biotin, MMP-1-inhibiting substances, and esters of retinol (vitamin A1) with a C₂₋₁₈ carboxylic acid.

28. The method of claim 25, wherein said composition comprises at least one additional substance, selected from the group consisting of surface active substances, amino acids, amino acid zinc salts, amino acid addition salts, film-forming polymers, emulsion-stabilizing polymers, thickening polymers, adhesive polymers, fats, surfactants, anti-perspirants and polyols, protein hydrolyzates, protein hydrolyzate derivatives, monosaccharides, oligosaccharides, polysaccharides, saccharide derivatives, α-hydroxycarboxylic acids, α-ketocarboxylic acids α-hydroxycarboxylic acid esters, α-hydroxycarboxylic acid lactones, salts of α-hydroxycarboxylic acid, α-ketocarboxylic acid esters, α-ketocarboxylic lactones, and salts of α-ketocarboxylic acid.

29. The method according to claim 28, wherein said composition comprises at least one additional substance, selected from the group consisting of surface active substances, amino acids, amino acid zinc salts, amino acid addition salts, film-forming polymers, emulsion-stabilizing polymers, thickening polymers, adhesive polymers, fats, surfactants, anti-perspirants and polyols, protein hydrolyzates, protein hydrolyzate derivatives, monosaccharides, oligosaccharides, polysaccharides, saccharide derivatives, α-hydroxycarboxylic acids, α-ketocarboxylic acids α-hydroxycarboxylic acid esters, α-hydroxycarboxylic acid lactones, salts of α-hydroxycarboxylic acid, α-ketocarboxylic acid esters, α-ketocarboxylic lactones, and salts of α-ketocarboxylic acid.

30. The method of claim 25, wherein said treatment is carried out preventatively.
